(19) United States
(12) Patent Application Publication
(10) Pub. No.: US 2020/0354562 A1
(43) Pub. Date: Nov. 12, 2020

(54) FILMS AND COMPOSITES AND METHODS OF PRODUCTION AND USE

(71) Applicant: Texas Tech University System, Lubbock, TX (US)

(72) Inventor: Sami Nazzal, Dallas, TX (US)

(21) Appl. No.: 16/965,434

(22) PCT Filed: Jan. 31, 2019
(86) PCT No.: PCT/US2019/016125

§ 371 Date: Jul. 28, 2020

Related U.S. Application Data
(60) Provisional application No. 62/624,608, filed on Jan. 31, 2018.

(51) Int. Cl. C08L 51/08; C09J 7/38; C09J 11/06; C09J 151/08; C08F 283/06; C08J 5/18; C08J 3/00; A61K 9/70; A61L 15/24; A61K 9/48; A61K 8/02; A61K 8/91; A61Q 19/00; A61K 9/28; A61K 8/67; A61L 15/20; A23L 27/20; A23L 27/10; A23L 27/00; A23P 20/20; B29C 39/12; B29C 39/00

(52) U.S. Cl. CPC C08L 51/08; B29K 2031/04; C09J 11/06; C09J 151/08; C08F 283/06; C08J 5/18; C08J 3/005; A61K 9/7023; A61L 15/24; A61K 9/4816; A61K 8/0212; A61K 8/91; A61Q 19/00; A61K 9/7007; A61K 9/284; A61K 9/282; A61K 8/678; A61L 15/20; A23L 27/202; A23L 27/10; A23L 27/74; A23L 27/79; A23P 20/20; B29C 39/123; B29C 39/003; C08L 2203/02; C08L 2203/16; C08J 2351/08; C08J 2491/00; A23V 2002/00; C09J 7/38

(57) ABSTRACT

The present invention includes compositions and methods of making films, adhesive patches, or composites comprising a polyvinyl caprolactam-polyvinyl acetate-polyethylene glycol graft copolymer (PCL-PVAc-PEG) and a partially hydrophilic oil, wherein the composition transitions from a viscous liquid, to an adhesive, and to a film as a weight percent (wt %) ratio of PCL-PVAc-PEG to partially hydrophilic oil changes.

### TECHNICAL FIELD OF THE INVENTION

The present invention relates in general to the field of novel adhesives, films, and methods of making and using the same.

### BACKGROUND OF THE INVENTION

Without limiting the scope of the invention, its background is described in connection with polymer mixtures.

Polymer mixtures and include, e.g., SOLUPLUS® (polyvinyl caprolactam-polyvinyl acetate-polyethylene glycol graft copolymer (PCL-PVAc-PEG)), which is an excipient that has been used in the formulation of drugs or active pharmaceutical ingredients (APIs) and the development of medicines. Soluplus® is also used in applications, including hot melt extrusion and electrospinning, to form orally available drugs where the solubility and dissolution rates of the API are dramatically improved. Historically, attempts at solvent casting Soluplus® have been met with difficulty, since the thin films are brittle and fracture easily. While development of pliable thin films would expand the use of this copolymer in drug development, the long felt need has yet to met. A need remains for novel biocompatible adhesives and films that can be customized for use in various applications.

### SUMMARY OF THE INVENTION

In one embodiment, the present invention includes a composition comprising a polyvinyl caprolactam-polyvinyl acetate-polyethylene glycol graft copolymer (PCL-PVAc-PEG) and a partially hydrophilic oil, wherein the composition transitions from a viscous liquid, to an adhesive patch, and to a film as a weight percent (wt %) ratio of PCL-PVAc-PEG to partially hydrophilic oil changes. In one aspect, the transition between the viscous liquid to the adhesive patch occurs when the PCL-PVAc-PEG comprises about 25 to 50 wt % and the partially hydrophilic oil comprises about 50 to 75 wt % of the composition. In another aspect, the transition between the adhesive and the film occurs when the PCL-PVAc-PEG comprises about 50 to less than 100 wt % and the partially hydrophilic oil comprises greater than 0% to 50 wt % of the composition. In another aspect, a tensile strength of the film ranges from 0.01 MPa to 6 MPa. In another aspect, an elasticity of the firm ranges from 50 MPa to 4500 MPa Young's modulus. In another aspect, an adhesiveness of the adhesive has a range from 6 g to 540 g of force. In another aspect, the PCL-PVAc-PEG has a molecular weight in the range of 90,000-140,000 g/mol. In another aspect, the partially hydrophilic oil has a free hydroxyl group. In another aspect, the partially hydrophilic oil is at least one of vitamin E, eugenol, clove oil, or black seed oil. In another aspect, the composition further comprises a polar solvent. In another aspect, the composition further comprises an active agent. In another aspect, the composition further comprises depositing or molding the composition into an adhesive, a film, a composite, an insect or rodent trap, a pressure sensitive adhesive, a transdermal drug delivery patch, a pressure sensitive adhesive, a transdermal drug delivery patch, a film, a pill coating, a wound dressing, a general adhesive, a glue, a food aid, a gummy, an edible film, a face mask, or a soft or a hard gelatin-free capsule.

In another embodiment, the present invention includes a method of making a composition that transitions between a viscous liquid, an adhesive patch, and a film comprising the steps of: mixing a acetate-polyethylene glycol graft copolymer (PCL-PVAc-PEG) and a partially hydrophilic oil; forming a homogeneous mixture; and depositing an adhesive patch or casting into a film. In one aspect, the method further comprises adding a polar solvent during the mixing step, wherein the polar solvent is one from the group of acetone, ethanol, methanol, and dimethylformamide. In another aspect, the method of mixing comprises at least one of sonication, vibration, vortexing, mixing, stirring, shaking, or heating. In another aspect, a film is cast by at least one or extrusion, solution casting, or reverse roll coating. In another aspect, the method further comprises removing any bubbles from the mixture. In another aspect, the transition between the viscous liquid to the adhesive patch occurs when the PCL-PVAc-PEG comprises about 25 to 50 wt % and the partially hydrophilic oil comprises about 50 to 75 wt % of the composition. In another aspect, the transition between the adhesive and the film occurs when the PCL-PVAc-PEG comprises about 50 to less than 100 wt % and the partially hydrophilic oil comprises greater than 0% to 50 wt % of the composition. In another aspect, a tensile strength of the film ranges from 0.01 MPa to 6 MPa. In another aspect, an elasticity of the firm ranges from 50 MPa to 4500 MPa Young's modulus. In another aspect, an adhesiveness of the adhesive has a range from 6 g to 540 g of force. In another aspect, the PCL-PVAc-PEG has a molecular weight in the range of 90,000-140,000 g/mol. In another aspect, the partially hydrophilic oil has a free hydroxyl group. In another aspect, the partially hydrophilic oil is at least one of vitamin E, eugenol, clove oil, or black seed oil. In another aspect, the method further comprises forming the composition in into an adhesive, a film, a composite, an insect or rodent trap, a pressure sensitive adhesive, a transdermal drug delivery patch, a pressure sensitive adhesive, a transdermal drug delivery patch, a film, a pill coating, a wound dressing, a general adhesive, a glue, a food aid, a gummy, an edible film, a face mask, or a soft or a hard gelatin-free capsule. In another aspect, the composition further comprises an active agent.

In another embodiment, the present invention includes a composition comprising: a polyvinyl caprolactam-polyvinyl acetate-polyethylene glycol graft copolymer (PCL-PVAc-PEG); and a partially hydrophilic oil, wherein the composition transitions from a viscous liquid, to an adhesive, and to a film as a weight percent (wt %) ratio of PCL-PVAc-PEG to partially hydrophilic oil changes, the transition between the viscous liquid to the adhesive occurs when the PCL-PVAc-PEG comprises about 25 to 50 wt % and the partially hydrophilic oil comprises about 50 to 75 wt % of the composition, and the transition between the adhesive and the film occurs when the PCL-PVAc-PEG comprises about 50 to less than 100 wt % and the partially hydrophilic oil comprises greater than 0% to 50 wt % of the composition. In one aspect, the composition is formed into an adhesive, a film, a composite, an insect or rodent trap, a pressure sensitive adhesive, a transdermal drug delivery patch, a pressure sensitive adhesive, a transdermal drug delivery patch, a film, a pill coating, a wound dressing, a general adhesive, a glue, a food aid, a gummy, an edible film, a face mask, or a soft or a hard gelatin-free capsule.

In another embodiment, the present invention includes a pressure sensitive adhesive, a transdermal drug delivery patch, a film, or a pill coating composition that transitions between a viscous liquid, an adhesive, and a film made by a method comprising: mixing a acetate-polyethylene glycol graft copolymer (PCL-PVAc-PEG) and a partially hydrophilic oil; forming a homogeneous mixture; and depositing an adhesive or casting solution into a film.

### BRIEF DESCRIPTION OF THE DRAWINGS

For a more complete understanding of the features and advantages of the present invention, reference is now made to the detailed description of the invention along with the accompanying figures and in which:

FIGS. 1A-1C show the chemical structures of (FIG. 1A) PCL-PVAc-PEG (specifically, Soluplus®) and (FIG. 1B) α-tocopherol (vitamin E), and FIG. 1C is a graphical abstract that shows the percentages of the oil and the PCL-PVAc-PEG and the properties of the mixes.

FIG. 2 is an image showing the consistency of pure α-tocopherol (vitamin E) and the viscous PCL-PVAc-PEG/Vitamin E composite at 25% w/w PCL-PVAc-PEG load.

FIGS. 3A to 3B show typical figures of (FIG. 3A) amplitude sweep curves for determining the LVR region and (FIG. 3B) force/time curve from the texture analysis test.

FIGS. 4A to 4D show FTIR spectra of (FIG. 4A) PCL-PVAc-PEG, (FIG. 4B) α-tocopherol (vitamin E), (FIG. 4C) PCL-PVAc-PEG/Vitamin E physical mixture at 15% w/w PCL-PVAc-PEG concentration in the mixture, and (FIG. 4D) PCL-PVAc-PEG/Vitamin E viscous solution at 15% w/w PCL-PVAc-PEG concentration in the composite.

FIGS. 5A and 5B show the correlation between shear rate and shear stress showing the effect of % w/w PCL-PVAc-PEG concentration on the (FIG. 5A) viscosity and (FIG. 5B) flow behavior of the PCL-PVAc-PEG/Vitamin E composites at 20±1° C.

FIGS. 6A and 6B show an amplitude sweep tests showing the effect of % w/w PCL-PVAc-PEG concentration on the (FIG. 6A) storage modulus (G′) and (FIG. 6B) loss modulus (G″) of the PCL-PVAc-PEG/Vitamin E composites at ω=6.283 rad/s, T=20±1° C.

FIGS. 7A and 7B show angular frequency tests showing the effect of % w/w PCL-PVAc-PEG concentration on the (FIG. 7A) storage (G′), (FIG. 7B) loss (G″) moduli, and tangent of the phase angle (tan δ) of the PCL-PVAc-PEG/Vitamin E composites at 20±1° C.

FIGS. 8A to 8C show temperature ramp tests showing the effect of temperature on the (FIG. 8A) storage modulus (G′), (FIG. 8B) loss (G″) modulus, and (FIG. 8C) viscosity (η′) of the PCL-PVAc-PEG/Vitamin E composites at different % w/w PCL-PVAc-PEG loading in the composites at a 6.283 rad/s angular frequency and 5 Pa oscillatory stress.

FIGS. 9A to 9C shows texture analysis showing the effect of % w/w PCL-PVAc-PEG concentration on (FIG. 9A) hardness, (FIG. 9B) adhesiveness and (FIG. 9C) cohesiveness effect of % w/w PCL-PVAc-PEG concentration at 20±1° C.

FIGS. 10A and 10B show: (FIG. 10A) ASTM dog bone punch (D-638-V) showing film dimensions and a sample of a 30% vitamin E film, and (FIG. 10B) a typical stress-strain curve for PCL-PVAc-PEG/vitamin E films undergoing tensile strain testing. This figure was obtained from a texture analysis of a 30% vitamin E film

FIGS. 11A to 11D show fourier transform infrared (FTIR) spectra of (FIG. 11A) PCL-PVAc-PEG film, (FIG. 11B) Vitamin E, (FIG. 11C) PCL-PVAc-PEG film with 30% vitamin E, and (FIG. 11D) PCL-PVAc-PEG film with 50% vitamin E

FIG. 12 shows dhermograms determined by MDSC showing the total heat flow of PCL-PVAc-PEG/vitamin E films containing different concentrations of vitamin E (0%-50% w/w)

FIGS. 13A to 13C show powder x-ray diffraction (PXRD) patterns of PCL-PVAc-PEG/vitamin E films containing (FIG. 13A) 0%, (FIG. 13B) 30% and (FIG. 13C) 50% vitamin E

FIGS. 14A and 14B show: (FIG. 14A) Tensile strength of PCL-PVAc-PEG films cast at a wet thickness of 20 mils containing different concentrations of vitamin E (0%-50% w/w), and (FIG. 14B) Tensile strength of 50% w/w vitamin E films that were cast at different wet thicknesses (1 mil=0.0254 mm)

FIGS. 15A and 15B show: (FIG. 15A) Percent elongation of PCL-PVAc-PEG films cast at a wet thickness of 20 mils containing different concentrations of vitamin E (0%-50% w/w), and (FIG. 15B) Percent elongation of 50% w/w vitamin E films that were cast at different wet thicknesses (1 mil=0.0254 mm). The asterisk (*) indicates that the films (30 and 40 mils) stretched the entire length of the tensile test (225 mm) without breaking

FIGS. 16A and 16B show: (FIG. 16A) Young modulus of PCL-PVAc-PEG films cast at a wet thickness of 20 mils containing different concentrations of vitamin E (0%-50% w/w), and (FIG. 16B) Young modulus of 50% w/w vitamin E films that were cast at different wet thicknesses (1 mil=0.0254 mm)

FIGS. 17A and 17B show: (FIG. 17A) Adhesiveness (tack) of PCL-PVAc-PEG films that were cast at a wet thickness of 20 mils and made with 0%-75% w/w vitamin E. Also shown in this figure is the adhesiveness of three grades of DURO-TAK® pressure sensitive adhesives (DURO-TAK® 87-900A, 87-2852, and 387-2510), which were referred to in the figure as DT1, DT2, and DT3, respectively, and (FIG. 17B) adhesiveness of the 50% w/w vitamin E films that were cast at different wet thicknesses (1 mil=0.0254 mm)

FIG. 18 shows peel adhesion strength of PCL-PVAc-PEG films that were cast at a wet thickness of 20 mils and made with 50%-75% w/w vitamin E (1 mil=0.0254 mm). Also shown in this figure is the peel adhesion strength of three grades of DUIRO-TAK® pressure sensitive adhesives (DURO-TAK® 87-900A, 87-2852, and 387-2510, which were referred to in the figure as DT1, DT2, and DT3, respectively. Asterisk (**) indicates that the films made with 65, 70, and 75% vitamin E left reside when peeled and therefore the peel adhesion test of these films may not accurately reflect their adhesion strength.

FIG. 19 shows water contact angle of the PCL-PVAc-PEG/vitamin E films as a function of vitamin E concentration (0-75% w/w).

FIG. 20 shows the Swelling capacity of PCL-PVAc-PEG/vitamin E films containing different concentrations of vitamin E (0-50% w/w). The asterisk (*) indicates that the 0% and 10% films dissolved or disintegrated in water, respectively, and therefore the swelling capacity of these films could not be measured.

FIG. 21 shows the disintegration of PCL-PVAc-PEG/vitamin E films in water as a function of time. Images were captured with a camera at time 0 (initial) for films containing 0-50% w/w vitamin E. The black line across the image is intended to show the transparency of the films when cast. Subsequent images show the films at different time points when immersed in water. Data from films immersed for 6 hours were used as the basis for the swelling study in FIG. 11.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the following terms are used. A “viscous liquid” refers to a material that is flowable at room temperature and that may or may not be tacky. An “adhesive patch” refers to a materials that are substantially non-flowable and that had adhesiveness, ranging from tacky to very tacky such as those material used for insect to rodent traps. A “film” as used herein refers to a material that is mostly solid and that may or may not be tacky. The films of the present invention can be adhesive and may stretch without losing integrity, similar to PARAFILM®. A film of the present invention can be ductile, malleable, waterproof, odorless, translucent and have cohesive thermoplastic properties. As used with the present invention, the polymeric portion of the composition is generally solid at room temperature and can be heated to cause the material to soften to the point of being a viscous liquid. By adding varying percentages of the partially hydrophilic oil and/or adding different combination of partially hydrophilic oils in different amounts, the composition can transition from the three basic states described hereinabove, namely, a viscous liquid to an adhesive patch to a film, with varying levels of tackiness or adhesiveness achieved by the change in weight percent of the polymeric material and the partially hydrophilic oil. Further, solvents can be added to increase the flowability of the material during certain phases for manufacturing purposes, and then the solvent can be withdrawn to reduce or generally eliminate the flowability of the final material, e.g., into a flexible or solid film.

As used herein, the term “active ingredient(s),” “pharmaceutical ingredient(s),” “active agents” and “bioactive agent” are defined as drugs and/or pharmaceutically active ingredients. The present invention may be used to encapsulate, attach, bind or otherwise be used to affect the storage, stability, longevity and/or release of any of the following drugs as the pharmaceutically active agent in a composition.

Non-limiting examples of active agents that may be included with, or delivered by, the compositions of the present invention include, but are not limited to, antibiotics, analgesics, vaccines, anticonvulsants; antidiabetic agents, antifungal agents, antineoplastic agents, antiparkinsonian agents, antirheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic compounds are water-soluble, poorly water-soluble drug or a drug with a low, medium or high melting point. The therapeutic compounds may be provided with or without a stabilizing salt or salts.

One or more of the following active agents may be combined with one or more carriers and the present invention (whether in the adhesive and/or the film form): analgesic anti-inflammatory agents such as, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

Drugs having an action on the central nervous system, for example sedatives, hypnotics, antianxiety agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocaine, benzocaine, fentanyl, nicotine, and the like.

Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, and the like. Anti-allergenics such as, antazoline, methapyrilene, chlorpheniramine, pyrilamine, pheniramine, and the like.

Decongestants such as phenylephrine, ephedrine, naphazoline, tetrahydrozoline, and the like.

Antipyretics such as aspirin, salicylamide, non-steroidal anti-inflammatory agents, and the like. Antimigrane agents such as, dihydroergotamine, pizotyline, and the like.

Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like.

Steroids such as, androgenic steriods, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β₂ -adrenergic agonists, such as, albuterol, terbutaline, metaproterenol, ritodrine, carbuterol, fenoterol, quinterenol, rimiterol, solmefamol, soterenol, tetroquinol, and the like.

Sympathomimetics such as, dopamine, norepinephrine, phenylpropanolamine, phenylephrine, pseudoephedrine, amphetamine, propylhexedrine, arecoline, and the like.

Local anesthetics such as, benzocaine, procaine, dibucaine, lidocaine, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE₁, PGE₂α, and PGF₂α, and the PGE₁ analog misoprostol. Antispasmodics such as, atropine, methantheline, papaverine, cinnamedrine, methscopolamine, and the like.

Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenytoin, and the like. Agents for dizziness such as, isoprenaline, betahistine, scopolamine, and the like. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like.

Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranquilizers such as, chlorprathixene, thiothixene, haloperidol, bromperidol, loxapine, and molindone, as well as, those agents used at lower doses in the treatment of nausea, vomiting, and the like.

Muscle relaxants such as, tolperisone, baclofen, dantrolene sodium, cyclobenzaprine.

Drugs for Parkinson's disease, spasticity, and acute muscle spasms such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like. Respiratory agents such as, codeine, ephedrine, isoproterenol, dextromethorphan, orciprenaline, ipratropium bromide, cromglycic acid, and the like. Non-steroidal hormones or antihormones such as, corticotropin, oxytocin, vasopressin, salivary hormone, thyroid hormone, adrenal hormone, kallikrein, insulin, oxendolone, and the like.

Vitamins such as, vitamins A, B, C, D, E and K and derivatives thereof, calciferols, mecobalamin, and the like for dermatologically use. Enzymes such as, lysozyme, urokinaze, and the like. Herb medicines or crude extracts such as, Aloe vera, and the like.

Antitumor agents such as, 5-fluorouracil and derivatives thereof, krestin, picibanil, ancitabine, cytarabine, and the like. Anti-estrogen or anti-hormone agents such as, tamoxifen or human chorionic gonadotropin, and the like. Miotics such as pilocarpine, and the like.

Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like.

Mydriatics such as, atropine, cyclopentolate, homatropine, scopolamine, tropicamide, eucatropine, hydroxyamphetamine, and the like. Psychic energizers such as 3-(2-aminopropy)indole, 3-(2-aminobutyl)indole, and the like.

Antidepressant drugs such as, isocarboxazid, phenelzine, tranylcypromine, imipramine, amitriptyline, trimipramine, doxepin, desipramine, nortriptyline, protriptyline, amoxapine, maprotiline, trazodone, and the like.

Anti-diabetics such as, insulin, and anticancer drugs such as, tamoxifen, methotrexate, and the like.

Anorectic drugs such as, dextroamphetamine, methamphetamine, phenylpropanolamine, fenfluramine, diethylpropion, mazindol, phentermine, and the like.

Anti-malarials such as, the 4-aminoquinolines, alphaaminoquinolines, chloroquine, pyrimethamine, and the like.

Anti-ulcerative agents such as, misoprostol, omeprazole, enprostil, and the like.

Antiulcer agents such as, allantoin, aldioxa, alcloxa, N-methylscopolamine methylsuflate, and the like. Antidiabetics such as insulin, and the like.

For use with vaccines, one or more antigens, such as, natural, heat-killer, inactivated, synthetic, peptides and even T cell epitopes (e.g., GADE, DAGE, MAGE, etc.) and the like.

The drugs mentioned above may be used in combination as required. Moreover, the above drugs may be used either in the free form or, if capable of forming salts, in the form of a salt with a suitable acid or base. If the drugs have a carboxyl group, their esters may be employed.

The acid mentioned above may be an organic acid, for example, methanesulfonic acid, lactic acid, tartaric acid, fumaric acid, maleic acid, acetic acid, or an inorganic acid, for example, hydrochloric acid, hydrobromic acid, phosphoric acid or sulfuric acid. The base may be an organic base, for example, ammonia, triethylamine, or an inorganic base, for example, sodium hydroxide or potassium hydroxide. The esters mentioned above may be alkyl esters, aryl esters, aralkyl esters, and the like.

When a drug different than an anesthetic agent is used the solvent selected is one in that the drug is soluble. In generally the polyhydric alcohol may be used as a solvent for a wide variety of drugs. Other useful solvents are those known to solubilize the drugs in question.

The present invention may be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a subject.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of the nanoshell composition as an active component and/or ingredient will be known to those of skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified.

The solid carrier may also include a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity may be maintained, for example, by the use of a coating, such as lecithin, by the maintenance of the required particle size in the case of dispersion and/or by the use of surfactants. The prevention of the action of microorganisms may be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions may be brought about by the use in the compositions of agents delaying absorption, for example, aluminum monostearate and/or gelatin.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like may also be employed.

Additional formulations that are suitable for other modes of administration include vaginal suppositories and/or suppositories. A rectal suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like using the present invention may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

The examples of pharmaceutical preparations described above are merely illustrative and not exhaustive. The adhesives and films of the present invention are amenable to most common pharmaceutical preparations.

### EXAMPLE 1

### The Rheological and Textural Characterization of Soluplus®/Vitamin E Composites

Graft amphiphilic copolymers are frequently used as an excipient in solid dosage forms as a dissolution and a solubility enhancer. The inventors discovered that one such graft amphiphilic copolymer polyvinyl caprolactam-polyvinyl acetate-polyethylene glycol graft copolymer (PCL-PVAc-PEG) (e.g., Soluplus®) can be dissolved in a partially hydrophilic oil, such as vitamin E, eugenol, or black seed oil. Interestingly, the PCL-PVAc-PEG was not soluble or interactive with other oils, such as olive oil. The result of the new formulation starts a tacky and highly adhesive material. The present inventors rheological, adhesive, and textural properties of the PCL-PVAc-PEG/partially hydrophilic oil composites is demonstrated. In one example, PCL-PVAc-PEG was dissolved under heat in vitamin E at increasing concentrations from 0 to 40% (by weight). The flow behavior of the PCL-PVAc-PEG/Vitamin E composites was determined by applying shear stress using an advanced AR2000 rheometer. Under the linear viscoelastic region (LVR), the rheological properties of the blends such as dynamic viscosity (η′), storage modulus (G′), loss modulus (G″), and the phase angle tangent (tan δ) were measured. Hardness, adhesiveness, and cohesiveness of the blends were also measured with a TA.XT plus texture analyzer. Rheological analysis showed that the viscosity of the PCL-PVAc-PEG/Vitamin E composites increased with an increase in PCL-PVAc-PEG concentration but decreased as the temperature increased from 20 to 90° C. The adhesiveness of the blends also significantly increased with an increase in PCL-PVAc-PEG concentration. The results from this study indicated that PCL-PVAc-PEG/Vitamin E composites have the potential to be exploited in applications where the use of highly adhesive material is desirable. Similar properties were obtained with the eugenol and/or black seed oil

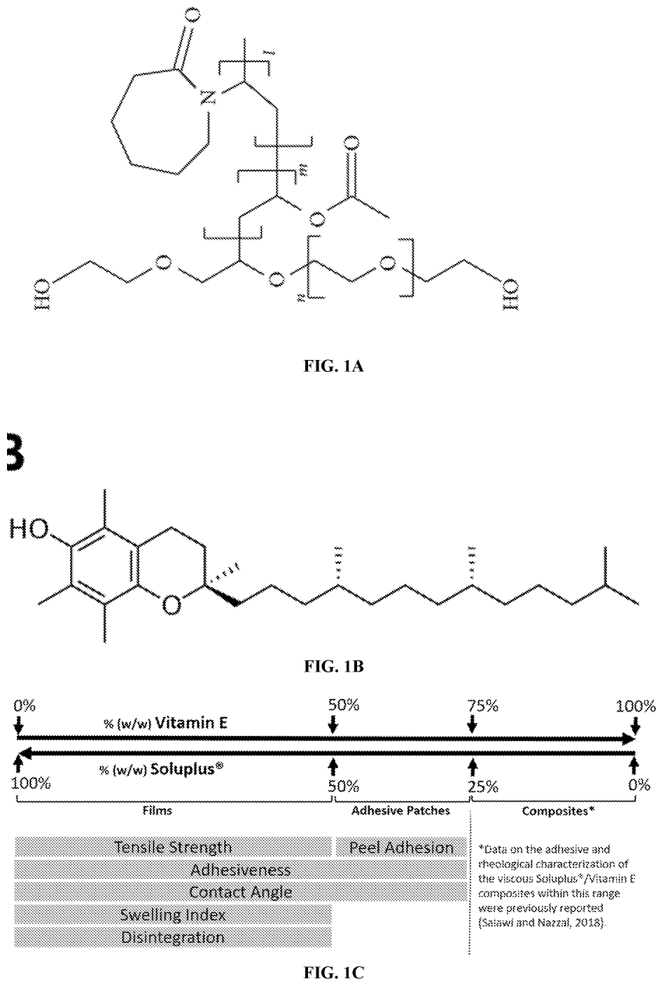

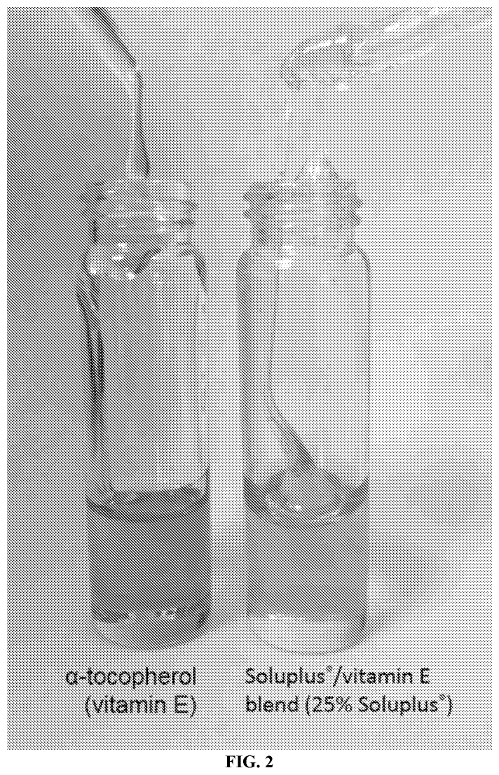

Amphiphilic high molecular weight polyvinyl caprolactam-polyvinyl acetate-polyethylene glycol grafted copolymer (PCL-PVAc-PEG) (mol. wt. 90,000-140,000 g/mol, FIG. 1A), such as Soluplus®, has a low glass transition temperature of 68° [1] and is supplied as white to slightly yellowish solid granules of low bulk density that have excellent flow properties. Recently, there has been a growing interest in the use of PCL-PVAc-PEG in pharmaceutical processes, most notably melt extrusion, spray drying, wet granulation, and direct compression because of its low hygroscopicity, glass transition temperature, and molten viscosity [2, 3].

The present inventors discovered that PCL-PVAc-PEG can dissolve in a select group of lipophilic non-volatile organic liquids, such as vitamin E and eugenol, producing tacky/viscous composites that may have pharmaceutical, food, cosmetic, and agricultural applications, to mention a few. Vitamin E, for example, may modulate the physical properties of PCL-PVAc-PEG and expand upon the uses of PCL-PVAc-PEG in melt extrusion. PCL-PVAc-PEG has already been investigated as a thickening agent for hydrophilic solvents, such as water, where it was found to significantly increase the viscosity and improve the elastic character of the solvents [4]. To the inventors' knowledge, the use of PCL-PVAc-PEG as a thickening agent for hydrophobic non-volatile organic solvents, has not been previously reported.

The rheological and textural properties of the PCL-PVAc-PEG/Vitamin E composites was evaluated. It was found that the viscosity of vitamin E can be significantly increased by the addition of PCL-PVAc-PEG. Furthermore, dispersing vitamin E in the PCL-PVAc-PEG network was also found to significantly increase the tackiness of the transparent liquid composite. This tackiness is an added advantage when surface adhesion is desirable, such as in glue and adhesion applications. This example shows the following: (1) measure and delineate the viscoelastic properties of the PCL-PVAc-PEG/Vitamin E composites, (2) examine the impact of temperature and angular frequency on their viscoelastic behavior, and (3) measure the adhesiveness of the composites as a function of polymer load by texture analysis.

Vitamin E is a liquid with a reported viscosity of 2480 centipoise at 24° C. [5]. Vitamin E refers to a family of eight related isomers which can be divided into tocopherol and tocotrienol subfamilies, with α-tocopherol (FIG. 1B) being the most abundant form. α-Tocopherol is an important fat-soluble antioxidant that is used in many cosmetic products for its anti-inflammatory effects on the skin. α-Tocopherol was shown to defend cell membranes and polyunsaturated lipids from ROS attack, and to protect the skin from harmful effects upon exposure to exogenous toxic agents such as pollutants, chemicals, and sun rays [6]. Vitamin E as a therapeutic agent and its composite with PCL-PVAc-PEG may therefore be used for wound healing and remedy for topical ailments [7-10]. Aside from its biological activity, vitamin E can also be used as a solvent for drugs [6, 11] for added benefits when the composite is used in topical cosmetic and/or pharmaceutical applications. FIG. 1C is a graphical abstract that shows the percentages of the oil and the PCL-PVAc-PEG and the properties of the mixes.

Materials. DL-α-Tocopherol (Vitamin E, >96% purity) was from TCI (Tokyo, Japan). Vitamin E acetate was from Alfa Aesar (Ward Hill, Mass., USA). The graft copolymer polyvinyl caprolactam-polyvinyl acetate-polyethylene glycol (PCL-PVAc-PEG, lot no. 84414368E0) was a generous gift from BASF (Ludwigshafen Germany).

Preparation of PCL-PVAc-PEG/Vitamin E composites. In glass vials, PCL-PVAc-PEG was mixed with vitamin E at 85° C. with the aid of a PowerMax 200 homogenizer (VWR International, Radnor, Pa., USA) until a clear amber colored solution was obtained. Composites containing PCL-PVAc-PEG at 5, 10, 15, 20 and 25% w/w concentrations were prepared. While it was possible to prepare composites containing up to 40% w/w PCL-PVAc-PEG, at concentrations ≥30% w/w the composites were too viscous to respond to applied force during rheological and mechanical testing. Therefore, these concentrations were excluded from further analysis. Composites were allowed to equilibrate to room temperature for 48 h before analysis. Visually, the difference in consistency between the pure vitamin E and the PCL-PVAc-PEG/Vitamin E composite at 25% w/w PCL-PVAc-PEG load can be seen in FIG. 2.

Fourier Transform Infrared (FT-IR) Spectroscopy. FT-IR spectra were used to characterize the possible interactions between PCL-PVAc-PEG and vitamin E. A PerkinElmer Spectrum Two™ spectrometer (Waltham, Mass.) attached to an attenuated total reflectance (ATR) accessory was used to collect spectral data of PCL-PVAc-PEG, vitamin E, and PCL-PVAc-PEG/Vitamin E composites and physical mixtures. Samples were directly placed on a diamond disk and scanned for absorbance within a wave number range from 800 to 4000 cm−1. Signal averages were obtained at a resolution of 4 cm−1. Three scans were used for each determination.

Rheological Characterization. PCL-PVAc-PEG/Vitamin E composites were expected to exhibit viscous and elastic behaviors. The viscoelastic properties of the composites were evaluated by measuring their shear rate under an applied shear stress. The viscous response, or the loss modulus, is the loss of the rigid structure of the system as a result of the applied stress over strain (G″). The recovery of the polymeric dispersions when lifting the applied stress is known as the elastic (G′) or storage modulus. Tangent of the phase angle (tan δ) is the ratio of the viscous property of the dispersion (G″) to its elastic property (G′). Tan δ is a measure of the relative contribution of the viscous component to the solid characteristics of the material [12]. Materials behave increasingly like a liquid as the tan δ value increases above 1, whereas they behave increasingly like a gel as the tan δ value decreases below 1 [13].

The characterization of the rheological properties of vitamin E and PCL-PVAc-PEG/Vitamin E composites was carried out with an Advanced AR 2000 controlled stress/controlled rate rheometer (TA Instruments, New Castle, Del., USA) with a 2o, 40 mm cone-plate geometry and a 61 μm gap between the plates. Approximately 0.6 mL of each sample was carefully placed on the lower plate. To ensure that sample shearing was minimized, samples were allowed to equilibrate for 5 min before analysis. Unless otherwise specified, at least three replicate analyses were carried out for each composite using a freshly prepared sample. Data analysis was performed using the Rheology Advantage™ software V5.8.2 (TA Instruments). All measurements were performed in triplicates.

Flow Rheometry Test. The flow behavior and complex viscosity (η) of the PCL-PVAc-PEG/Vitamin E composites were measured in flow mode at 20±1° C. under an applied shear stress from 0.60 to 59.68 Pa. This range was based on the strength of resistance to the applied stresses. Stress-strain curves were subsequently generated by the Rheology Advantage™ software.

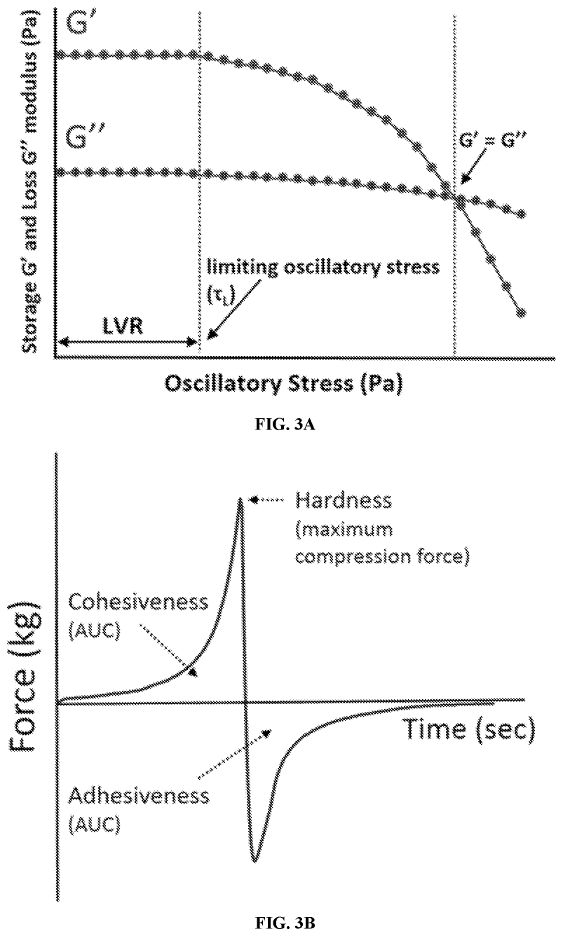

Amplitude Sweep Test. The amplitude sweep test was carried out to determine the range of the linear viscoelastic region (LVE) of the samples during which the measured storage modulus (G′) and loss modulus (G″) maintain a constant plateau value, i.e., the sample structure is conserved (FIG. 3A). During the amplitude sweep test, samples were exposed to an increasing oscillatory stress from 0.60 to 59.68 Pa, in a logarithmic ramp profile, while the angular frequency and temperature were kept constant (ω=6.283 rad/s, T=20±1° C.). The upper limit of oscillatory stress (τ) within the LVE region is known as the limiting oscillatory stress (τL). When the oscillatory stress exceeds τL, a change in G′ and G″ curves begins to be observed when plotted against oscillatory stress [12, 14-16] and the structure of the sample is irreversibly changed or completely ruined [12, 17, 18].

Angular Frequency Sweep Test. The angular frequency sweep test, which is commonly used to determine the viscoelastic properties of materials [19], was conducted on the samples over the angular frequency range from 6.283 to 62.830 rad/s and at a constant oscillatory stress of 5 Pa. An oscillatory stress of 5 Pa was selected because it resides within the LVE region and below the γL value of the samples. All measurements were carried out at 20±1° C. G′, G″, and the tangent of the phase angle (tan δ) were plotted as a function of angular frequency and analyzed with the aid of Rheology Advantage™ software.

Temperature Ramp Test. An oscillatory temperature ramp analysis was performed under a constant oscillatory stress of 5 Pa for each sample over a temperature range of 20 to 90° C. The temperature was controlled with a Peltier plate temperature system (TA Instruments, New Castle, Del., USA). Samples were analyzed at a 6.283 rad/s angular frequency and a 5° C./min heating rate. G′, G″ and η′ were plotted as a function of temperature and analyzed with the aid of Rheology Advantage™ software.

Texture Analysis. Texture analysis is a technique that has been widely utilized for the mechanical characterization of food and pharmaceutical products [20, 21]. The mechanical characteristics of the PCL-PVAc-PEG/Vitamin E composites were determined by using a TA-XT plus texture analyzer (Texture Technologies Corp and Stable Micro Systems, Ltd, Scarsdale, N.Y., USA) equipped with a 50 kg load cell and fitted with a 35 mm flat-faced stainless-steel cylindrical probe. Briefly, 10 g sample of each composite was placed in a 25 mL jacketed reaction beaker. Before analysis, air bubbles were removed from the samples by maintaining the reaction beaker at 80° C. for approximately 30 minutes. Samples were then allowed to equilibrate to room temperature for 24 h. After equilibration, samples were tested for their texture properties by compressing the probe into the surface of each sample at a rate of 2 mm/s to a depth of 4 mm, after which the probe was retracted at a rate of 2 mm/s. Composite Hardness, adhesiveness, and cohesiveness were then estimated from the generated force-time plots. At least three replicate analyses were carried out for each composite using a freshly prepared sample. Data collection and calculation were determined by the Texture Exponent 6.1.7.0 software package. A typical force-time plot is given in FIG. 3B.

Preparation of PCL-PVAc-PEG/Vitamin E composites. It has been reported that at 85° C., which is about 13° C. higher than the glass transition temperature of PCL-PVAc-PEG (Tg=72° C.), the tangent of the phase angle (tan δ) would equal 1 [13]. Heating PCL-PVAc-PEG to a temperature >85° C. is therefore required for its viscous (loss modulus) component to become dominant. Consequently, to obtain a clear and homogenous composite, PCL-PVAc-PEG and Vitamin E blends should be processed at ≥90° C., otherwise translucent/hazy dispersions would be obtained if the composites were prepared at lower temperatures.

FT-IR Spectroscopy. FTIR was employed to study PCL-PVAc-PEG-vitamin E interactions. A change in FTIR absorbance, such as a shift in the wavenumber or a change in peak intensity of the PCL-PVAc-PEG/Vitamin E composites when compared to the neat polymer or the PCL-PVAc-PEG/Vitamin E physical mixtures that were prepared without heating may provide valuable information on the possible interaction between the ingredients of the composites. Hydrogen bonding between PCL-PVAc-PEG and vitamin E was expected because it was observed in preliminary studies that PCL-PVAc-PEG does not dissolve under the same preparation conditions in vitamin E acetate, which lacks the free OH group in the chroman ring that is necessary for H-bonding.

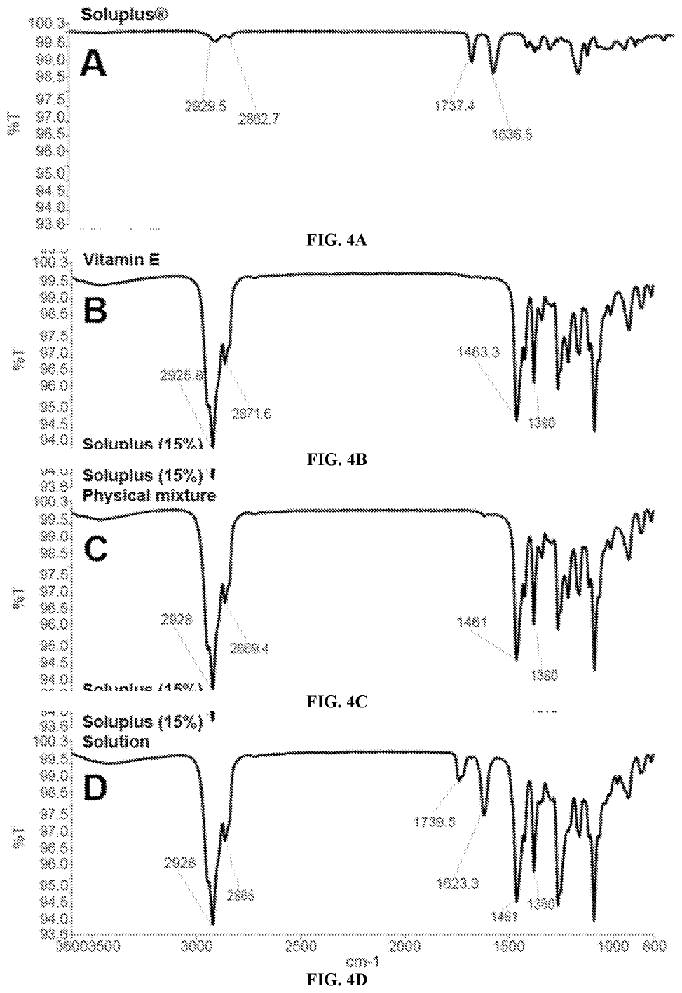

The presence of hydrogen bonding in the PCL-PVAc-PEG/Vitamin E composite was confirmed by comparing the FTIR spectra of the composites with those of the neat polymer and physical mixtures (FIG. 4). The FT-IR spectra of PCL-PVAc-PEG (FIG. 4A) revealed a characteristic peak at 2929.5 cm−1, which corresponds to —CH2— stretching. Similarly, for pure vitamin E (FIG. 4B), the bands at 2925.8 and 2871.6 cm−1 represent the asymmetric and symmetric stretching vibrations of the —CH2— and —CH3 groups, respectively. The important band of PCL-PVAc-PEG at 1636.5 cm−1 (C═O) increased in intensity in the PCL-PVAc-PEG/Vitamin E composite (FIG. 4D) when compared to neat Soluplus. This increase can be attributed to the presence of the intermolecular H-bonds between the carbonyl groups of PCL-PVAc-PEG (H-bond acceptor) and the hydroxyl groups (—OH) of vitamin E (H-bond donor). In addition, the stretching vibration of C═O of PCL-PVAc-PEG at 1636.5 cm−1 was shifted to a lower wavenumber (1623.3 cm−1) of a PCL-PVAc-PEG/Vitamin E composite confirming this interaction. No similar changes in peak intensity were observed in the physical mixture (FIG. 4C)

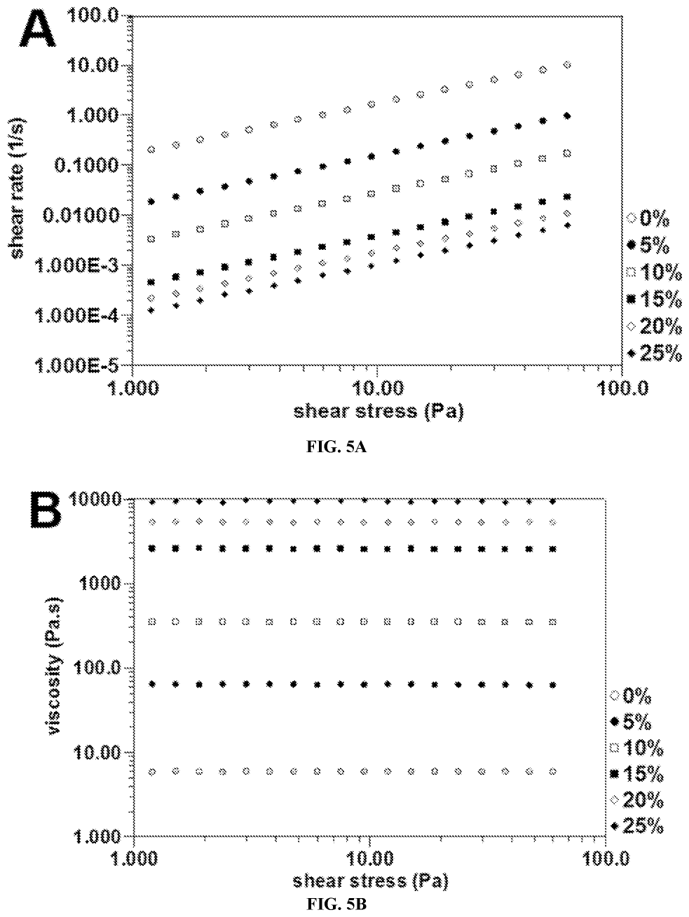

Rheological Characterization. Flow Rheometry Test. The flow profiles of the PCL-PVAc-PEG/Vitamin E composites showing the relationship between shear stress and shear rate under steady shear are given in FIG. 5A. It can be observed that shear stress and shear rate are proportional in all composites and show no yield stress with a PCL-PVAc-PEG concentration load of 0-25% (by weight). The proportionality of the shear stress and shear rate is typical of Newtonian behavior as shown by the independence of the viscosity of the composites with increasing shear stress (FIG. 5B). Newtonian behavior can be explained by the favorable association of PCL-PVAc-PEG with vitamin E, which helps avoid the gelling and aggregation of the polymer. The viscosity of the composites however was dependent on the concentration of PCL-PVAc-PEG. Viscosity increased from 5.9 Pa·s for pure vitamin E to approximately 10,000 Pa·s at 25% w/w PCL-PVAc-PEG load in the composite (FIG. 5B). The increase in viscosity could be attributed to the high molecular weight and the polymeric nature of PCL-PVAc-PEG, and the interaction between polymer chains with the solute under the applied stress [12].

Amplitude Sweep Test. The amplitude sweep test determines the viscoelastic properties of formulations by subjecting the samples to a sinusoidal oscillatory stress at a low oscillatory angle to avoid damaging the polymeric structure of the composites during measurements. From this test, two dynamic moduli were obtained. The storage modulus (G′), which is a measure of energy stored and recovered per deformation cycle, and the loss modulus (G″), which is a measure of the energy dissipated per cycle. G′ reflects the solid-like component of a viscoelastic material whereas G″ reflects the viscous or liquid-like component.

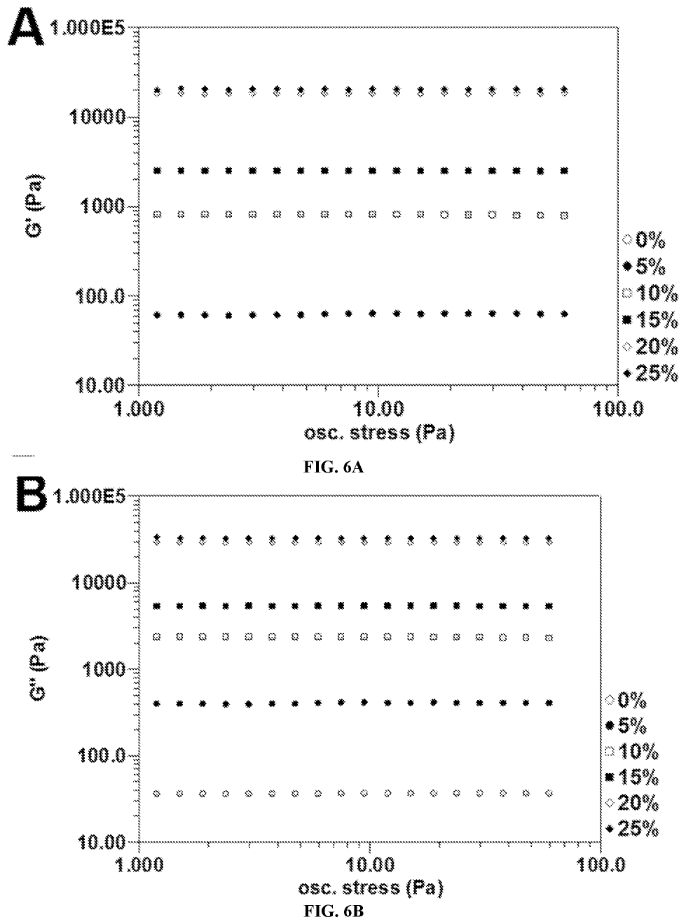

When plotted as a function of oscillatory stress, the G′ (FIG. 6A) and G″ curves (FIG. 6B) for all ratios—except for the pure vitamin E (0% PCL-PVAc-PEG) for which storage modulus could not be detected—were found to be oscillatory stress independent. Therefore, no limited oscillatory stress (γL) could be identified within the tested parameters. This indicated that no significant alteration or distortion to the internal structure can be noted within this range. Thus, a constant oscillatory stress of 5 Pa was selected when carrying out the subsequent angular frequency sweep and ramp temperature tests since a distortion to the internal structure of the composites was not expected at this value.

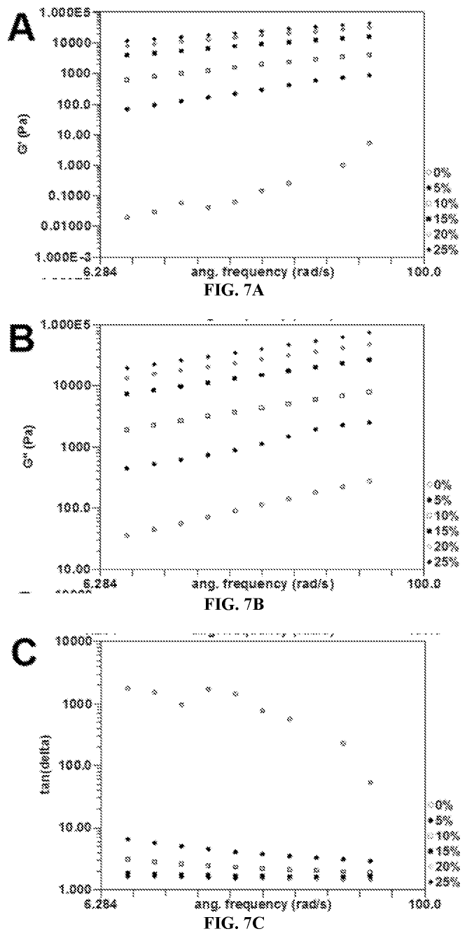

Angular Frequency Sweep Test. The effect of PCL-PVAc-PEG load in the composites on G′, G″ and tan δ as a function of angular frequency at a constant oscillatory stress of 5 Pa are shown in FIGS. 7A, 7B and 7C, respectively. The viscoelastic properties of the PCL-PVAc-PEG/Vitamin E composites were found to be angular frequency dependent. Increasing angular frequency increased G′ and G″, while, an increase in angular frequency decreased tan δ value. This effect was minimized by increasing a PCL-PVAc-PEG concentration. In all composites, no crossover between G′ and G″ was observed. Tan δ remained >1, where G″ values were greater than G′ values over the entire range of frequencies and concentrations, indicating that the composites had a liquid like behavior. This was expected as the flow rheometry measurements showed that the composites were mainly Newtonian (FIGS. 3 and 4). Although G″ remained greater than G′ at all frequencies, the rate of G′ increase was greater than G″, therefore the interval (tan δ) between G′ and G″ decreased as the PCL-PVAc-PEG concentration increased. The effect of PCL-PVAc-PEG on the viscoelastic behavior of vitamin E was unlike those observed in oleogel systems. In oleogles, a crossover between G′ and G″ was observed with increasing oscillatory stress indicating the formation of a gel like material [22]. PCL-PVAc-PEG/Vitamin E composites do not form gels; rather they retain their liquid like behavior, even at high PCL-PVAc-PEG loads.

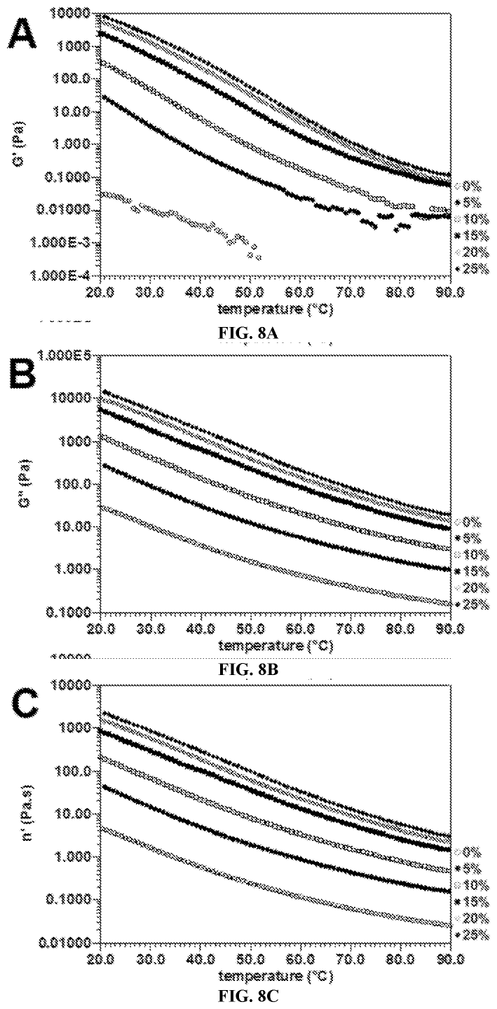

Temperature Ramp Test. The effect of temperature within a range from 20 to 90° C. on the thickening behavior of the PCL-PVAc-PEG/Vitamin E composites was evaluated using the temperature ramp test (FIG. 8). As shown in the figure, the viscoelastic properties of the composites were found to be temperature dependent. G′, G″, and η′ (FIGS. 8A, 8B, and 8C, respectively) decreased with an increase in temperature. The rate of change in the viscoelastic properties, however, was independent of the PCL-PVAc-PEG concentration. All composites showed similar responses to the temperature ramp as observed by the relatively parallel lines. This observation is typical of the fluid like behavior of materials, where G″ is greater than G′, with no observed crossover.

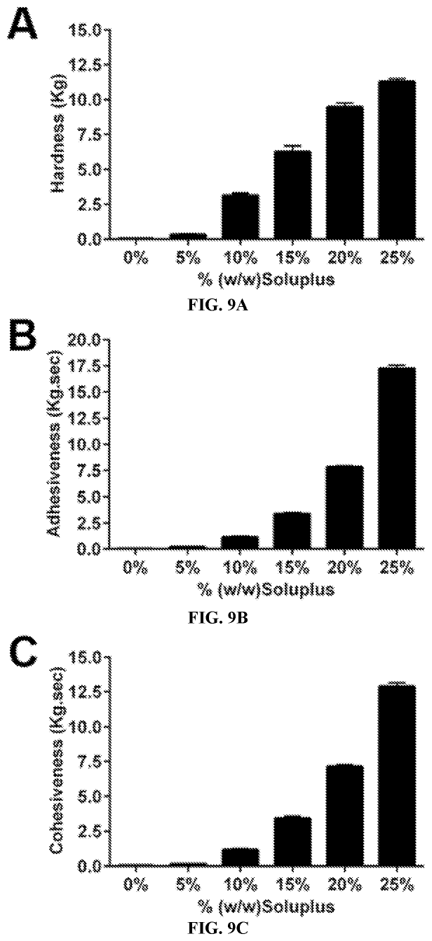

Texture Analysis. Texture profile analysis (TPA) of the PCL-PVAc-PEG/Vitamin E composites was carried out to study their compressional flow properties. A typical force/time profile that was generated from a TPA is shown in FIG. 2B [23]. It provided valuable information about the impact of PCL-PVAc-PEG load on the hardness, adhesiveness, and cohesiveness of the composites (FIG. 9). These characteristics quantify sample deformation under compression. Hardness describes the stress/work required to remove the sample from the container and to subsequently apply it to the site of application. It is defined as the force required to acquire a given deformation which is the same as the maximum peak force during compression. Hardness should be low in order to allow the material to be easily removed from the container. Adhesiveness is defined as the negative force area for the compression cycle and represents the work required to overcome the attractive forces between the surface of the adhesive formulation and the surface of the probe. Adhesiveness of materials is important as it has been correlated with clinical performance, where higher adhesiveness ensures better adhesion at topical surfaces and allows for prolonged retention time [24-26] [14, 27, 28]. The work required to deform the formulations in the down movement of the probe is a measure of cohesiveness [23].

The hardness, cohesiveness and adhesiveness of the composites increased with an increase in PCL-PVAc-PEG concentration in the composites from 0% to 25%. The hardness of the composites (FIG. 9A) increased from 0.021 kg for the pure vitamin E, to 11.49 kg for composites with 25% w/w PCL-PVAc-PEG. The increase in hardness, as a function of the PCL-PVAc-PEG concentration, was in agreement with previous studies which illustrated the dependence of hardness on polymer content [29]. As with hardness, adhesiveness (FIG. 9B) increased with an increase in the viscosity of the composites [30]. It has been reported that materials with a higher elastic component (G′) usually possess greater adhesion, which correspond to higher detachment forces [31]. This phenomenon was observed with an increase in the PCL-PVAc-PEG concentration, where an increase in the adhesiveness of the composites, from 0.01 to 17.63 kg·sec, was observed with an increase in PCL-PVAc-PEG concentration from 0% to 25%.

The cohesiveness of the composites (FIG. 9C) is related to their internal restructuring during the application of force or pressure. At higher PCL-PVAc-PEG concentrations, stronger attractive forces between the polymer and vitamin E in the composites are expected which may explain the observed increase in cohesiveness from 0.02 to 13.17 kg·sec and the increase in PCL-PVAc-PEG concentration from 0% to 25%. A similar impact of polymer concentration on hardness, adhesive, and cohesive properties of gels was observed with hydroxypropyl methylcellulose and Carbopol [32, 33].

This example described the development and characterization of viscous PCL-PVAc-PEG/Vitamin E composites. The rheological (flow, amplitude sweep, oscillatory frequency, and ramp temperature) and mechanical (hardness, adhesiveness, and cohesiveness) tests provided valuable information on the viscoelastic and textural properties of the PCL-PVAc-PEG/Vitamin E composites can be used in pharmaceutical, food, industrial, and cosmetic products. This may include, but not limited to their topical use for dental, cosmetic and wound healing applications; as adhesives and traps in industrial products; as extruding agents, and potentially carriers for drugs for transdermal drug delivery applications. The viscosity of the composites was found to increase as a function of the PCL-PVAc-PEG concentration and to decrease with temperature. Unlike oleogels however, no G′/G″ crossover was observed, indicating that PCL-PVAc-PEG based dispersions in hydrophobic nonvolatile organic solvents retain their fluid like behavior even at high polymer loads. PCL-PVAc-PEG/Vitamin E composites were found to have high adhesive properties, which make them interesting systems as viscoelastic adhesive formulations for use in applications such as extending drug residence time at application site. The use of PCL-PVAc-PEG as a thickening agent is unique. Based on these results, PCL-PVAc-PEG was found to only disperse in select oils like vitamin E, eugenol, or black seed oil, which are partially hydrophilic and/or comprise a hydroxyl group.

### EXAMPLE 2

### The Physiochemical, Mechanical, and Adhesive Properties of Solvent-Cast Vitamin E/PCL-PVAc-PEG Films

Polyvinyl caprolactam-polyvinyl acetate-polyethylene glycol graft copolymer (PCL-PVAc-PEG) is an amphiphilic graft copolymer used in hot melt extrusion applications and electrospinning. One example of a PCL-PVAc-PEG is Soluplus® (BASF) which is an excipient and was used for all the examples herein. Very little information is available on the use of PCL-PVAc-PEG as a film former and in the development of film-based formulations. The overall aim of this work was to study the mechanical and adhesive properties of PCL-PVAc-PEG films prepared by the solvent casting technique. More specifically, the inventors discovered that vitamin E can serve as a plasticizer for the PCL-PVAc-PEG polymer and to significantly modulate its mechanical and adhesive properties. Vitamin E (0-75% w/w) and PCL-PVAc-PEG were dissolved in ethanol and cast on liners to produce transparent films. Cast films were tested for their physiochemical properties by IR, XRD, and MDSC, and for their adhesive and mechanical properties by texture analysis. Vitamin E was found to be miscible with PCL-PVAc-PEG and to reduce the crystallinity of the films. Vitamin E also decreased the films' tensile strength and Young's modulus while significantly increasing their percent elongation. The most notable effect was the observed increase in the adhesiveness (tackiness) and hydrophobicity of the films, which was evidenced by a significant increase in their water contact angle and a decrease in their swelling capacity and disintegration. These observations indicated that vitamin E/PCL-PVAc-PEG blends might be used for the preparation of highly pliable films, especially when made with 30-50% vitamin E, and in the development of a new type of pressure sensitive adhesive films when prepared with ≥65% vitamin E load.

Polymeric films made from natural or synthetic polymers are used in a wide variety of food (Dirim et al., 2004), cosmetic (Kaji et al., 2017), and pharmaceutical applications (Banker, 1966; Ofori-Kwakye and Fell, 2003). They could be prepared from either solvent or water based dispersions by various methods including spray coating and solvent casting where the evaporation of the solvent from a solution or dispersion leaves a continuous layer of polymeric film (Boateng et al., 2009). However, the mechanical properties of films that have been prepared by either methods may impact their handling and utility, primarily when used in pharmaceutical and clinical applications (Boateng et al., 2009). For example, hard and brittle films that have been prepared for use as dressings for topical applications may damage delicate and newly formed tissues around a wound leading to prolonged wound-healing times and the associated inconvenience to both patients and clinicians (Boateng et al., 2009). Therefore, plasticizers are often added to polymeric dispersions to improve the physical and mechanical properties of films (Lim and Hoag, 2013). Plasticizers are low molecular weight compounds that increase the free volume between the polymer chains, thereby lowering the polymer glass transition temperature and increasing the percent elongation of cast films (Lim and Hoag, 2013).

Due to its amphiphilic properties, PCL-PVAc-PEG, a polyvinyl caprolactam-polyvinyl acetate-polyethylene glycol grafted copolymer, has been widely used as a polymeric solubilizer in hot melt extrusion (HME) and electrospinning applications to enhance the dissolution rate of drugs (Shamma and Basha, 2013). Recently, PCL-PVAc-PEG was evaluated as a film forming material in coating applications. The utility of PCL-PVAc-PEG as a film former, however, was found to be limited due to the brittleness of the solvent-cast films, which were prepared from neat PCL-PVAc-PEG without the addition of plasticizers (Lim and Hoag, 2013). This limitation was readily resolved by the use of water-soluble plasticizers, such as triethyl citrate (TEC) and glycerin, which significantly improved the mechanical properties of PCL-PVAc-PEG films and enhanced their elongation capacity (Lim and Hoag, 2013).

In the example above, the inventors demonstrated that several lipophilic nonvolatile organic solvents, such as vitamin E, clove oil, eugenol, and black seed oil, could also be used to plasticize PCL-PVAc-PEG yielding highly flexible and transparent films. When the PCL-PVAc-PEG/vitamin E films were prepared by the solvent casting method the inventors also found that the amount of vitamin E in the blend has a significant effect on the adhesive properties of the films. Composites made with high vitamin E concentrations, for example, could potentially be used as pressure sensitive adhesives.

The objective of this study was to present preliminary data on the physiochemical, mechanical, and adhesive properties of solvent-cast films made from the binary PCL-PVAc-PEG/vitamin E blends at 100/0 to 25/75 ratios by weight. A detailed characterization of the viscous blends made with ≥75% vitamin E was previously reported (Salawi and Nazzal, 2018). In this this study, drug-free cast films were characterized by texture analysis, thermal analysis, x-ray diffraction, and IR spectroscopy, disintegration, swelling capacity, and contact angle. The results from this study highlighted the unique properties of the PCL-PVAc-PEG/vitamin E films, demonstrating the potential use in the development of pharmaceutical, cosmetic, industrial, or food products. Vitamin E, used as a plasticizer and main component in these films, also presents a unique opportunity to develop innovative products owing to its inherent therapeutic benefits. Vitamin E, an important fat-soluble antioxidant, has been used in cosmetic products due to its anti-inflammatory effects on the skin and its protective properties against harmful exposure to exogenous toxic agents such as pollutants, chemicals and sun rays (Boscoboinik et al., 1991; Cassano, 2012).

Materials. DL-α-Tocopherol (Vitamin E, >96% purity) was from TCI (Portland, Oreg., USA). PCL-PVAc-PEG was a generous gift from BASF (Ludwigshafen Germany). Ethanol was from Pharmco-AAPER (Brookfield, Conn., USA). DURO-TAK® (87-900A, 87-2852, and 387-2510) pressure sensitive adhesives were from Henkel Corporation (Bridgewater, N.J., USA). Scotchpak™ 1022 release liner and Scotchpak™ 1109 backing tan polyester film were from 3M (Oakdale, Minn., USA).

Preparation of PCL-PVAc-PEG/vitamin E films. PCL-PVAc-PEG films, with different weight ratios of vitamin E were prepared using the solvent casting technique. Briefly, 3 g PCL-PVAc-PEG was dissolved in 5 mL ethanol, with the aid of a PowerMax 200 homogenizer (VWR International, Radnor, Pa., USA), until a clear solution was obtained. Vitamin E then was added to the ethanolic PCL-PVAc-PEG solution to get a final concentration of 10%, 20%, 30%, 40%, 50%, 55%, 60%, 65%, 70%, and 75% w/w of vitamin E (calculated as percentage of vitamin E in the dry PCL-PVAc-PEG/vitamin E blend). The mixing continued for an additional 5 minutes. The PCL-PVAc-PEG/vitamin E solutions were then sonicated using a 5510R-DTH sonicator bath (Parasonic Ultrasonic Corporation, Danbury, Conn., USA) to remove air bubbles, for approximately 5-10 minutes. Solutions, containing up to 75% w/w vitamin E, were then cast with an 8-path film applicator onto a Scotchpak™ 1022 release liner to a wet thickness of 20 mils (1 mil=0.0254 mm). To evaluate the effect of film thickness on mechanical properties, the blend with 50% w/w vitamin E was cast to a wet thickness of 15, 20, 25, 30, and 40 mils. After casting, films were dried at ambient conditions for 24 hours and then under vacuum for an additional 48 h to ensure complete removal of solvent. After drying, the film thickness was measured at five different points with a digital micrometer. For composites containing ≥50% vitamin E, a Scotchpak™ 1109 backing layer was applied on the film with a 4.5 pound roller and conditioned for 24 hours at room temperature before being used in subsequent analysis. Films containing up to 50% vitamin E by weight were tested for their tensile strength, whereas cast films containing 50-75% of vitamin E by weight underwent peel adhesion test as described below.

Fourier Transform Infrared (FT-IR) Spectroscopy. FT-IR spectra were used to characterize the possible interactions between PCL-PVAc-PEG and vitamin E. A PerkinElmer Spectrum Two™ spectrometer (Waltham, Mass.) attached to an attenuated total reflectance (ATR) accessory was used to collect spectral data of films containing 0, 30, and 50% vitamin E by weight. Film samples were directly placed on the diamond disk of the ATR accessory and scanned for absorbance, within a wave number range from 800 to 4000 cm−1. Signal averages were obtained at a resolution of 4 cm−1. Three scans were used for each determination.

Thermal analysis. Modulated differential scanning calorimetry (MDSC) was conducted on films containing 0-50% vitamin E by weight, using a TA 2920 MDSC (TA Instruments-Waters LLC., New Castle, Del.) equipped with a refrigerated cooling system. Samples (5 mg) from each cast film were accurately weighed in aluminum pans and placed in a vacuum chamber overnight (Sheldon Manufacturing, Inc., Cornelius, Oreg). The pans were then hermetically sealed and heated from −50° C. to 140° C., at a rate of 2° C./min and modulation amplitude of 1° C. per 40 s, under a continuous nitrogen flow. Data were generated and analyzed by the TA Universal Analysis 2000 Software.

Powder X-ray diffraction (PXRD). The PXRD patterns of the 0%, 30% and 50% w/w/vitamin E films were collected by a Bruker D8 Discover Multi-Function X-Ray Diffractometer equipped with a Vantec-500 detector and a monochromatic Cu Kα radiation X-Ray tube radiation (λ=1.54056 Å). Films were cut into approximately 3×3 cm specimens, placed on a silicone plate, and fitted into the sample metal holder. Samples were scanned between 10° and 85° 2θ. All patterns were obtained at 25° C. using a voltage and current of 40 kV and 40 mA. Data were generated and analyzed by DIFFRAC.EVA (Bruker).

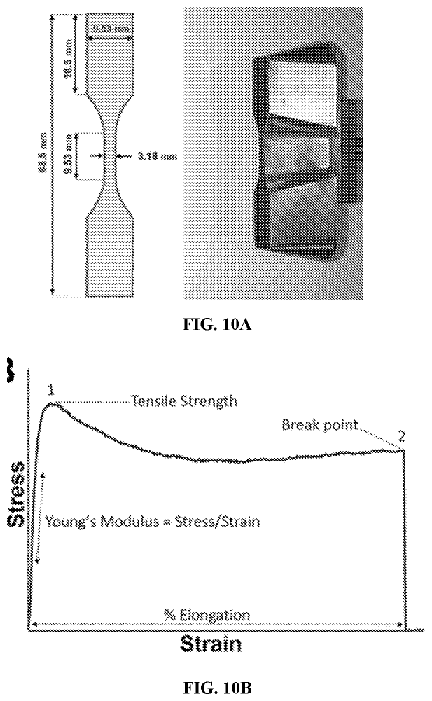

Mechanical characterization of cast films. Tensile strength test. The cast films containing 0%-50% vitamin E by weight were cut with a special die (Qualitest, Fla., USA) to conform to ASTM D638 type V dog bone shape (FIG. 10A). Films with misappropriated sides, cracks, or air bubbles were discarded. The mechanical properties of the films were measured using a texture analyzer, model TA-XT plus (Texture Technologies Corp and Stable Micro Systems, Ltd, Scarsdale, N.Y.) equipped with a 50 kg load cell. Film specimens were held between the grips of a TA-96B miniature tensile probe. To measure the tensile strength of the films, the grips of the tensile probe were allowed to move apart at a speed of 5 mm/sec to a target distance of 225 mm. The load and displacement data were recorded by the exponent stable micro system software v 6.1.7.0. Data that were generated from films that broke at the center of the dog bone shape were used to construct stress-strain curves, while data from films that broke near the grips were rejected. Tensile strength, percent elongation, and Young's modulus of each film were extrapolated from the stress-strain curves as previously described (Thakhiew et al., 2013). Briefly, the tensile strength, which is the force at which the film fractures, was obtained using the following relationship where Fₘₐₓ is the load at failure, t is the initial film thickness, and w is the initial film width:

$$Tensile trength = \frac{{F}_{max}}{t \times w} \qquad ( 1 )$$

The percent elongation was estimated from the film's strain at break as follows:

$$\begin{matrix}\% elongation = strain at break \times 100 & ( 2 ) \\ Strain at break = \frac{{l}_{f \_} {l}_{0}}{{l}_{0}} & ( 3 )\end{matrix}$$

l_{f} is the final length of the film at failure and l₀ is the initial length of the film between grips.

Young's modulus was obtained from the slope of the initial linear section of the stress strain curve where the film withstands elastic deformation (Lim and Hoag, 2013; Thakhiew et al., 2013).

Adhesiveness (tackiness) test. The adhesiveness or tackiness of the PCL-PVAc-PEG/vitamin E films (0%-75% w/w vitamin E) was measured using the TA-XT plus texture analyzer, fitted with a TA-58 flat-faced stainless-steel probe (8 mm diameter, 35 mm long). Cast films were firmly secured between two brushed aluminum plates. The upper plate facing the probe had 9 mm diameter holes that allow the probe to adhere to the exposed surface of the film. During the test, the probe was lowered at a speed of 0.1 mm/s into the hole. When the probe reached a trigger force of 5 gm, upon contact with the film, it was held in place for 5 seconds then allowed to retract at a speed of 0.5 mm/s. The force/time plots that were generated by the exponent 6.1.7.0 software were used to calculate adhesive force; a measure of the adhesive strength of the films.

180 degree Peel adhesion test. Peel adhesion measures the force required to peel away an adhesive once it has been attached to a surface. The peel adhesion test was developed for industrial tapes and subsequently adopted for transdermal delivery systems (patches). This test was carried out by the TA-XT plus texture analyzer fitted with a 180 degree peel adhesion assembly. The peel adhesion test was only used for cast films that contained 50-75% vitamin E by weight. At these ratios, cast films formed an adhesive layer that could not be readily peeled from the backing layer. Instead they were cut into 2.5 cm wide adhesive films or patches. Films were then applied to a stainless-steel plate and smoothed with a 4.5 pound roller and then pulled from the plates at a 180 angle at a rate of 300 mm/min. The generated force/displacement plots were used to calculate the peel adhesion strength.

Contact angle measurement. The contact angle of films, containing 0%-75% vitamin E, was measured in the air at room temperature using a static contact angle goniometer (OCA 15/20, Future Digital Scientific Corp., N.Y., USA). A 2 μL drop of distilled water was deposited by a syringe on the surface of the films. The image and shape of the drop was captured by the instrument and used to calculate the contact angle (θ). Two measurements were made for each film, and the mean value was calculated.

Swelling index. The water absorption capacity or the swelling index of films is a test that was developed to determine their usefulness in biomedical applications (Baskar and Sampath Kumar, 2009). The test was carried out on films containing 0%-50% vitamin E by weight in distilled water. Films were first cut into circles (1″ diameter) with a hollow puncher (Mayhew Pro™, Turners Falls, Mass.). Specimens were then weighed (W1) and placed in separate glass Petri dishes containing 50 mL of distilled water. The dishes were stored at room temperature. After 6 hours, the films were removed and the excess water on the surface was carefully removed by blotting with lint-free Kimwipes tissue papers. The swollen films were weighed again (W2), and the percentage swelling (swelling index, SI) was calculated as follows:

$$SI ( \% ) = ( \frac{W 2 - W 1}{W 1} ) \times 100 \qquad ( 4 )$$

Disintegration test. Cast films, containing 0%-50% vitamin E by weight, were cut into circles (1″ diameter) with a hollow puncher and placed in separate glass Petri dishes containing 50 mL of distilled water. The dishes were allowed to shake at room temperature in a VWR© incubating microplate shaker overnight at a constant speed of 100 rpm. Films were visually inspected, and photographs were taken at different time intervals up to 48 h. The time-lapsed composite image of the films was subsequently used to assess their dissolution and/or disintegration behavior.

Preparation of PCL-PVAc-PEG/vitamin E films. It is demonstrated herein that PCL-PVAc-PEG could be admixed with vitamin E to produce viscous composites, pressure sensitive adhesives, or transparent films depending on the ratio of the two ingredients in the blend and the means by which they were prepared. A detailed characterization of the viscous blend's composites, made with ≥75% vitamin E, was previously reported (Salawi and Nazzal, 2018). In the present example, films prepared by casting PCL-PVAc-PEG/vitamin E blends, made with up to 75% vitamin E by weight, were characterized for their mechanical and adhesive properties. It was found that transparent and highly pliable films could be readily made by the solvent casting method from blends containing up to 50% w/w vitamin E. When cast at wet thickness of 20 mils, the average thickness of the 0, 10, 20, 30, 40, 50, 55, 60, 65, 70, and 75% vitamin E films was 0.08, 0.09, 0.09, 0.102, 0.103, 0.105, 0.108, 0.109, 0.114, 0.117, and 0.119 mm, respectively.

The thickness of the dry films that were prepared at a wet thickness of 15, 20, 25, 30, and 40 mils was 0.12, 0.38, 0.41, 0.48, and 0.53 mm, respectively. At vitamin E concentrations >50%, however, cast films could not be readily peeled off from the release liner without breaking and therefore they were not tested for their tensile strength by a texture analyzer. Instead, a backing membrane was applied on the films to form a strong bond and to make it easy to remove the release liner. The residual adhesive layer on the backing membrane was tested for its tackiness and peel adhesion strength for potential use as a pressure sensitive adhesive.

Physiochemical characterization of films. FTIR was employed to study the possible interaction between PCL-PVAc-PEG and vitamin E in the cast films. A change in FTIR absorbance, such as a shift in the wavenumber or a change in peak intensity for the composites that contained vitamin E when compared to the neat PCL-PVAc-PEG film may provide valuable information on the possible interaction between the ingredients of the films. Hydrogen bonding between PCL-PVAc-PEG and vitamin E in the film samples, was expected because a similar interaction had been previously observed in the viscous PCL-PVAc-PEG blends, at high vitamin E loads (Salawi and Nazzal, 2018).

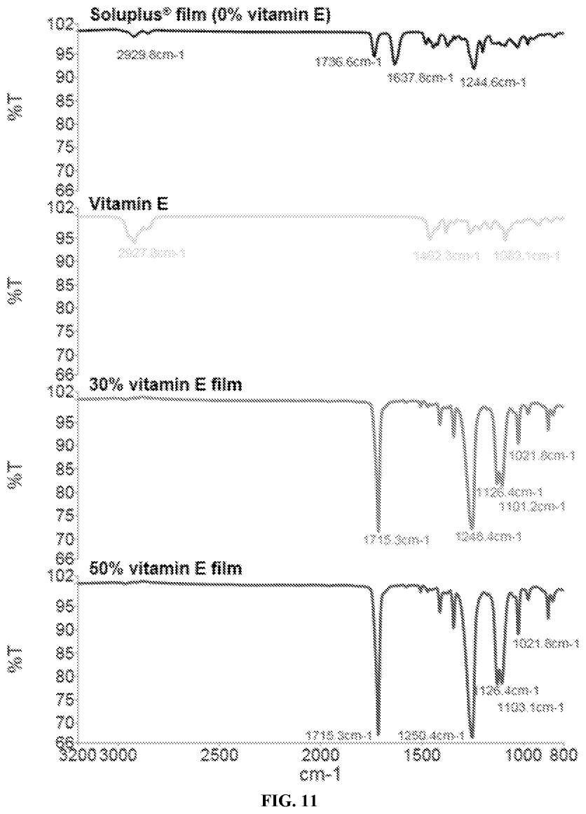

The presence of hydrogen bonding in the films was confirmed by comparing the FTIR spectra of the neat PCL-PVAc-PEG films with films containing vitamin E (FIG. 11). The FT-IR spectra of the neat PCL-PVAc-PEG film revealed a characteristic peak at 2929.8 cm−1, which corresponds to —CH2—. At 30 and 50% vitamin E load, the bands at 2929.8 disappeared due to the presence of vitamin E in the films while the PCL-PVAc-PEG band at 1736.6 cm−1 (C═O) increased in intensity. This increase can be attributed to the presence of the intermolecular H-bonds between the carbonyl groups of PCL-PVAc-PEG (H-bond acceptor) and the hydroxyl groups (—OH) of vitamin E (H-bond donor). Also, the stretching vibration of C═O of PCL-PVAc-PEG at 1736.6 cm−1 was shifted to a lower wavenumber (1715.3 cm−1), confirming this interaction (Jog et al., 2016; Salawi and Nazzal, 2018).

In the solvent casting process, the low rate of solvent evaporation may also lead to the crystallization of polymer molecules in the cast film, as they slowly diffuse to the crystal growth front and overcome the energy barrier of deposition (Hsu and Lawrence Yao, 2014). This could be readily seen in the thermal graph of the neat PCL-PVAc-PEG film (FIG. 12), that revealed a sharp endothermic peak at 49.50° C., indicating crystallinity of the cast film. A gradual shift in the crystallinity of the film to an amorphous state was observed when the concentration of vitamin E increased in the film. This intensity of the PCL-PVAc-PEG melting endotherm decreased with an increase in vitamin E concertation in the film from 10% to 30% and disappeared completely at 40%. The thermal transition of the films, with an increase in vitamin E concentration, could be attributed to the disruption of the ordered structure or crystalline state of the cast film to an amorphous state. These thermal results also support the plasticizing effect of Vitamin E and its effect on decreasing the brittleness of the films as reported later.

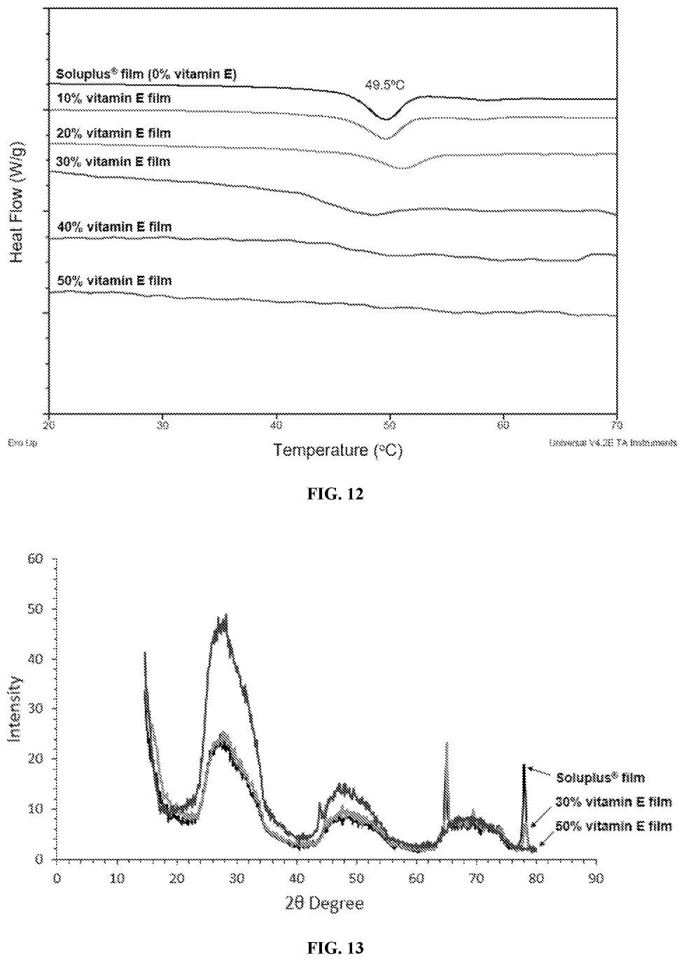

The absence of crystallinity can be confirmed by spotting the pattern in the X-ray diffractogram. PXRD of neat PCL-PVAc-PEG film and film containing 30% vitamin E showed sharp and narrow peaks at an angle of 20 of 65 and 78 degrees (FIG. 13). This indicated that the films made of neat PCL-PVAc-PEG or with vitamin E, up to 30% films, were crystalline in nature. The 30% vitamin E film nonetheless exhibited decreasing crystallinity as manifested by a decrease in intensity of the peaks at a 20 angle of 78 degrees. On the other hand, the PXRD pattern of the 50% vitamin E film indicated an amorphous state as it showed no peaks (FIG. 13). These observations confirmed the data that were generated from thermal analysis that showed a gradual decline in crystallinity of the films, with an increase in vitamin E load. PXRD and thermal data also support the observations that were generated from the mechanical testing of the films that demonstrated that films up top 30% were brittle while films made with higher vitamin E were increasingly elastic.

Mechanical characterization of films. Tensile test. When a strain was applied to the type V dog-bone shaped films (FIG. 10A) at a constant rate, the films extended until they tore at mid-point. A typical stress-strain curve generated from this test is shown in FIG. 10B, where points 1 and 2 are the film's tensile strength and break point (film fracture point), respectively. From this graph, the tensile strength, elongation, and Young's modulus were quantified and subsequently used to compare between the PCL-PVAc-PEG/vitamin E films.

The maximum tensile strength of a film is the maximum stress that a film can resist being stretched before failing or necking (Lim and Hoag, 2013). Plasticizers are often added to polymeric films to extend the free volume between the polymer chains. This addition leads to greater film flexibility and chain mobility (Felton et al., 2008). Consequently, a plasticized polymer would be less resilient and would deform at a lower force than without the plasticizer (Felton et al., 2008). The tensile strength of the PCL-PVAc-PEG/vitamin E films at fracture, as a function of vitamin E concentration, is shown in FIG. 14A. Films made with neat (100%) PCL-PVAc-PEG were brittle. As shown later, they had negligible elongation qualities and would readily fracture. Due to their brittle nature, they also had high tensile strength (11.61 MPa). The addition of vitamin E lowered the tensile strength of the films. This result was consistent with the general expectation for a plasticized polymeric film (Blanco-Fernandez et al., 2013; Lim and Hoag, 2013). As the vitamin E concentration increased from 10% to 50%, tensile strength significantly decreased from an average of 5.87 MPa to 0.24 MPa. For comparison, and to get a better feel for their mechanical properties, the tensile strength of the films made with 40% and 50% vitamin E were found to be similar to the tensile strength of Parafilm® “M” films. The increase in films thickness was also found to reduce tensile strength (FIG. 14B). Films prepared at wet thickness of 30 and 40 mils were very elastic and would not break within the testing parameters. They had extremely low tensile strength of 0.16 and 0.09 MPa, respectively, and high elasticity as displayed by their high elongation.

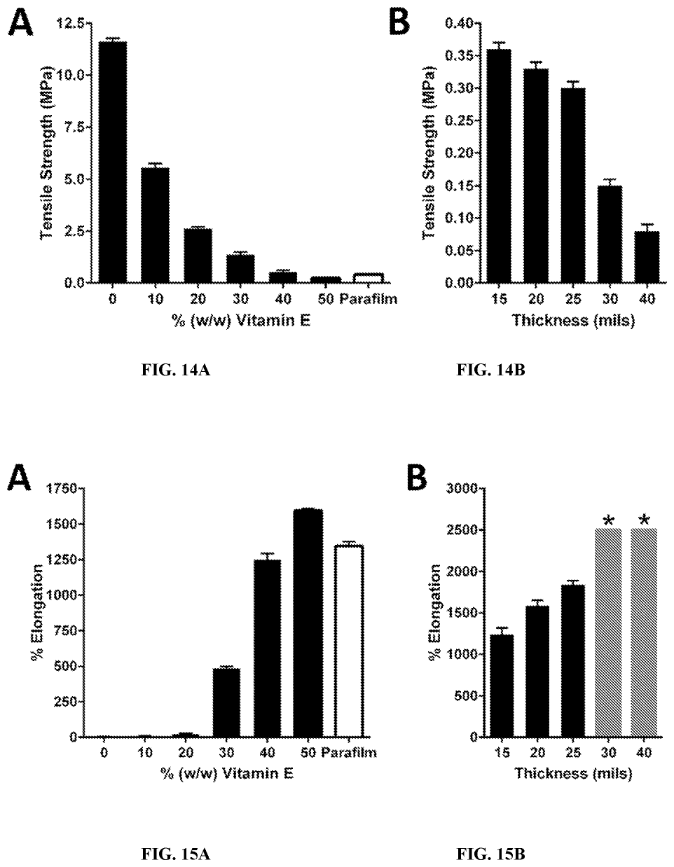

Percent elongation is a useful parameter for assessing the plasticizing effect of vitamin E, where elongation should increase with increasing plasticizer concentration (Aulton et al., 1995; Lim and Hoag, 2013). As shown in FIG. 15A, the percent elongation of the PCL-PVAc-PEG/vitamin E films increased with an increase in vitamin E concentration in the blend from almost 0% elongation for the neat PCL-PVAc-PEG film to nearly 1600% for films made with 50% vitamin E. The increase in elongation, however, was marginal for films made with up to 20% vitamin E. A significant increase in elongation was observed when the % vitamin E in the films was ≥30%. Films made with 40% and 50% vitamin E were similar in their elongation to the elongation property of the reference Parafilm® sample. Increasing wet thickness of the films also increased their elasticity (FIG. 15B). However, films prepared at a wet thickness of 30 and 40 mils were extremely elastic and stretched the length of the tensile test (225 mm) without breaking. Therefore, no percent elongation could be measured for these films within the testing limitation of the tensile test in this study.

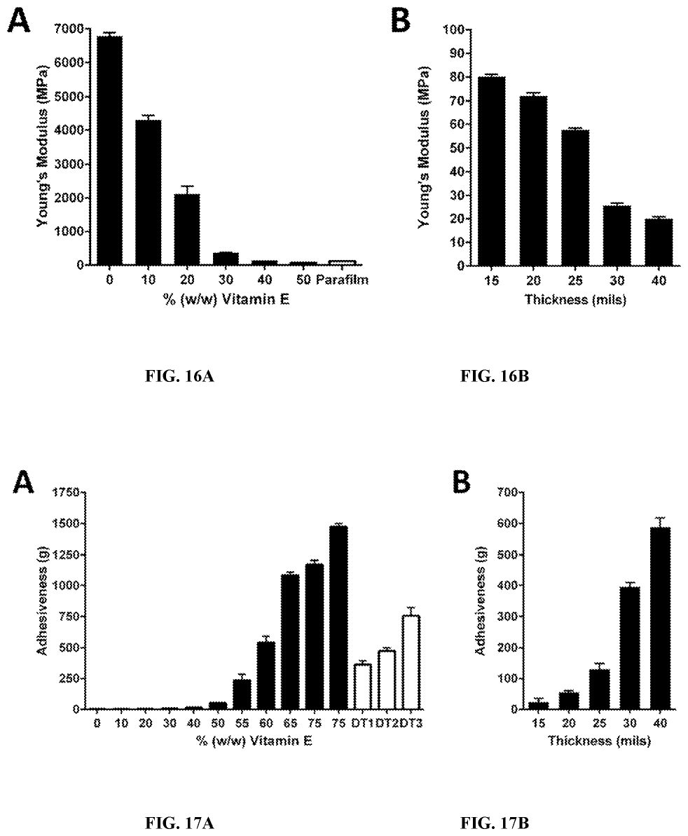

Young's modulus or modulus of elasticity, is the slope of the linear section of the stress-strain curve where the film withstands elastic deformation (Lim and Hoag, 2013). It measures the resistance of the films to plastic deformation, which can be used to indicate the strength and stiffness of the film (Felton et al., 2008; Karki et al., 2016). The higher values of Young's modulus correlate with stiffer films where higher loads are needed to cause elastic deformation (Aulton et al., 1995). The lower Young's modulus indicates flexible films where it needs lower loads to elastically deform (Banker, 1966; Felton et al., 2008). FIG. 16A shows Young's modulus of the PCL-PVAc-PEG films with different concentrations of vitamin E. Neat PCL-PVAc-PEG films were the stiffest (6787.46 MPa). Young's modulus subsequently decreased, with an increase in vitamin E concentration in the blend. The Young's modulus of films made with 10 and 20% vitamin E, was 4297.33 MPa and 2116.21 MPa, respectively. Although they had a lower Young's modulus and lower tensile strength than the neat PCL-PVAc-PEG film, they were stiff and showed similar mechanical behavior. A significant deviation in the mechanical properties of the films was observed when they were made with ≥30% vitamin E. The considerable decrease in Young's modulus indicated an increase in the elasticity of these films, which showed a higher percent elongation than films made with up to 20% vitamin E. Films with high vitamin E load were extremely elastic as indicated by their low Young's modulus of 136.5 MPa for the 40% vitamin E film and 68.14 MPa for the 50% vitamin E film (FIG. 16A). These films had similar elasticity to the reference Parafilm® sample. A similar trend in Young's modulus was also observed with an increase in film thickness, especially when prepared at a wet thickness of ≥30 mils.

Adhesiveness (tackiness) test. In the tack test, the adhesiveness of a film is measured as the force required to detach the texture probe from the film (Michaelis et al., 2014). The adhesiveness of the PCL-PVAc-PEG/vitamin E composites with 0-75% vitamin E load by weight is shown in FIG. 17A. An increase in vitamin E concentration significantly increased the adhesiveness or tack of the cast films. Films made with only PCL-PVAc-PEG had a negligible adhesion of approximately 1.19 g, which only slightly increased to 6.18 g for films made with 20% vitamin E. The most notable increase in adhesion was observed when the concentration of vitamin E was ≥50%. In perspective, the adhesion of films made from three grades of DURO-TAK® (87-900A, 87-2852, and 387-2510) was also measured. DURO-TAK® is a solvent-based acrylates copolymer dispersion that is used to prepare pressure sensitive drug-in-adhesive patches (Ahn et al., 2013; Anders and Lee, 2015; Jung et al., 2015; Schulz et al., 2010; Tuntiyasawasdikul et al., 2015). The three grades of DURO-TAK® that were used in this study were selected for their broad tackiness and peel adhesion strength. Other grades are also available that could be tested in future studies (Henkel, 2013). DURO-TAK® films were prepared by the casting method using the same procedure that was used for making the PCL-PVAc-PEG films at 20 mils. As seen in FIG. 17A, cast films made with >65% vitamin E had higher adhesiveness than the three DURO-TAK® grades. A similar increase in adhesiveness was also observed when the wet thickness of the 50% films was increased (FIG. 17B). Adhesiveness increased from 23.58 gm to 594.47 gm as the wet thickness of the films increased from 15 mils to 40 mils.

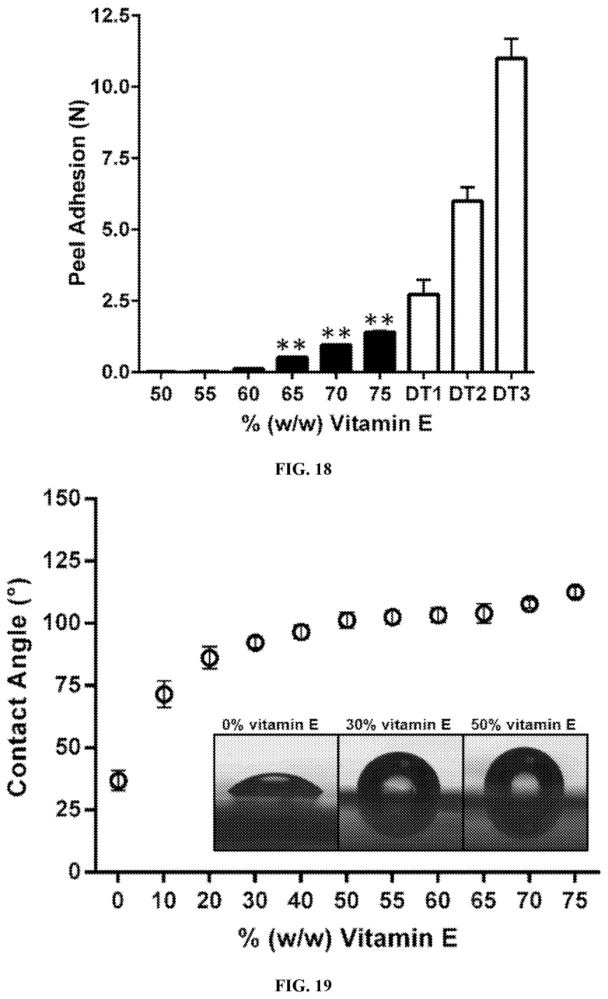

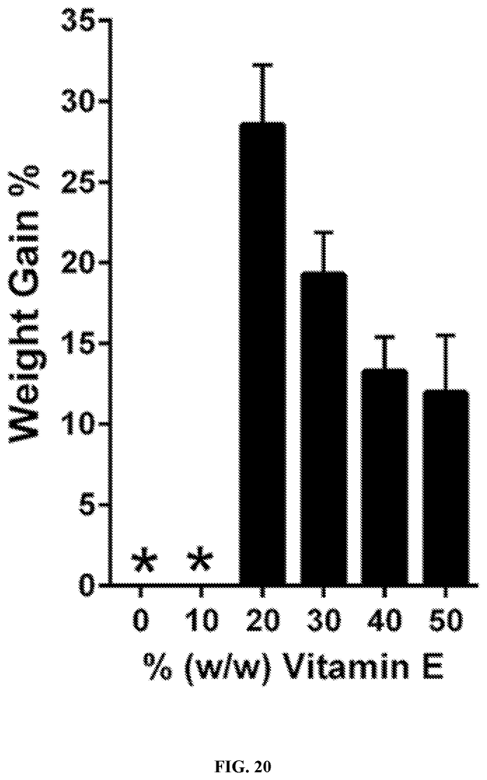

Peel Adhesion test. The peel adhesion test further demonstrated the adhesion properties of composites made with ≥50% vitamin E. Unlike the adhesiveness (tackiness) test, peel-adhesion test measured the force required to detach the adhesive films or patch from the stainless-steel surface at a 180-degree angle. The value of the adhesive force needed to detach the adhesive films from the application plate are given in FIG. 18. Composites with ≥65% vitamin E strongly adhered to the substrate and were difficult to peel without leaving a residue. On the other hand, films made with 50, 55 and 60% w/w vitamin E easily peeled from the test plate without leaving a residue, which could be due to their weak adhesion properties. Although the peel adhesion strength of these films was significantly less than the strength of the DURO-TAK® films, the results from this study when combined with the mechanical properties of the films and their tackiness highlighted the potential uses of the composites, especially at vitamin E concentrations ≥65% as pressure sensitive adhesives for topical applications and in other industrial applications such as insect traps or hair removal strips.

Wettability, swelling capacity, and disintegration of films. The wettability of the PCL-PVAc-PEG/vitamin E film surfaces was evaluated by measuring the static contact angles for water. The change in water contact angle values as a function of vitamin E concentration in the films is shown in FIG. 19. The wettability of film surfaces is mainly influenced by the degree of surface hydrophilicity, which explains the observed increase in contact angle with an increase in the concentration of the hydrophobic vitamin E in the films. For example, the contact angles of water droplets on the surface of neat PCL-PVAc-PEG film (0% vitamin E) and films made with 50% vitamin E was 37.3o and 104.7o, respectively. The impact of the hydrophobic surface of the films was apparent in their behavior upon exposure to moisture and their disintegration pattern.

The swelling capacity of films, which is related to their hydrophilic nature (Hermans et al., 2014) plays a vital role in film retention and the release of bioactive molecules for drug loaded films. When visually inspected, it was found that the swelling of the PCL-PVAc-PEG/vitamin E films was dependent on vitamin E concentration in the films as shown in FIG. 11. Films made from neat PCL-PVAc-PEG and with 10% vitamin E readily dissolved or disintegrated in water, respectively, and therefore the swelling capacity of these films could not be measured. Films made with ≥20% vitamin E remained intact. The 20% vitamin E films which had the highest PCL-PVAc-PEG load had the highest water uptake (28.5%). The swelling capacity of the films subsequently decreased with an increase in the concentration of the hydrophobic vitamin E in the blend to a low of 10.6% for the 50% vitamin E film.

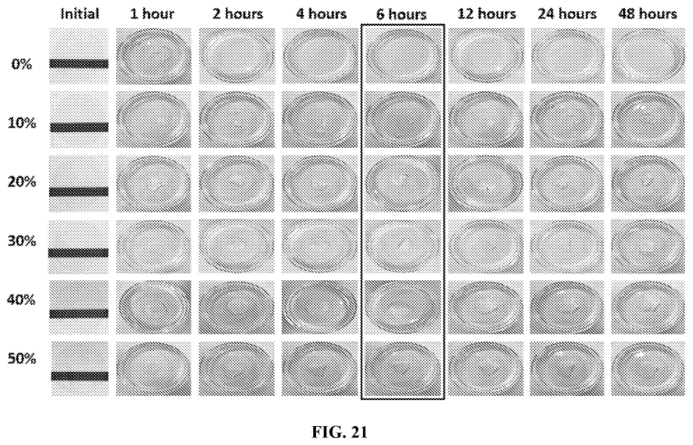

Disintegration test was carried out to visually observe the time-lapsed behavior of the PCL-PVAc-PEG/vitamin E films when immersed in water. All films were intact and transparent before the commencement of the study. At different time intervals during the test, photos were taken of the films as shown in FIG. 21. Of the films, only the hydrophilic neat PCL-PVAc-PEG film (0%) quickly dissolved within one hour. With an increase in vitamin E concentration, the films became increasingly resistant to disintegration and dissolution. The 10% vitamin E film disintegrated and broke into smaller fragments within less than 6 hours, which confirms the data observed in the swelling index study. Films made with ≥20% vitamin E remained mostly intact. They did not disintegrate and retained their structure for up to 48 hours. All films, nonetheless, became translucent/hazy upon exposure to moisture.

The present invention shows that vitamin E can be used as a plasticizer in PCL-PVAc-PEG dispersions to prepare transparent and highly elastic and adhesive films. Increasing vitamin E concentration in the PCL-PVAc-PEG/vitamin E films from 0%, for neat PCL-PVAc-PEG film, to 50% was found to decrease their crystallinity, tensile strength and elastic modulus and increase their percent elongation, indicating an increase in the flexibility and stretching of the films. Increasing vitamin E concentration in the films was also found to increase their adhesiveness. Cast films were very tacky and could not be readily removed from the backing layer, especially at vitamin E concentrations ≥65% by weight, without leaving a residue. Films made with 30% vitamin E could be considered as the point where significant changes in film properties was observed. A significant change in the mechanical properties of the films was also observed as a function of film thickness, where the increase in films thickness was found to increase their elasticity and adhesiveness. Data from this study indicated that the PCL-PVAc-PEG/vitamin E films may have unique applications that may not be realized by the currently available pressure sensitive adhesives.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES—EXAMPLE 1

1. Khanfar, M., et al., Enhancement of the dissolution and bioavailability from freeze-dried powder of a hypocholesterolemic drug in the presence of Soluplus. Powder Technology, 2018. 329: p. 25-32.
2. Rehab N. Shamma, M. B., Soluplus®: A novel polymeric solubilizer for optimization of Carvedilol solid dispersions: Formulation design and effect of method of preparation. Powder Technology, 2013: p. 406-414.
3. H. Hardung, D. D., and S. Ali, Combining HME and solubilization: Soluplus®—The solid solution Drug Del. Tech, 2010(10).
4. Cespi, M., et al., Rheological characterization of polyvinyl caprolactam-polyvinyl acetate-polyethylene glycol graft copolymer (Soluplus®) water dispersions. Colloid and Polymer Science, 2014. 292(1): p. 235-241.
5. Yoshizumi, M. O., et al., Physical properties of vitamin E oil and silicone oil. Retina, 1985. 5(3): p. 163-7.
6. Cassano, R., Vitamin E chemistry, biological activity and benefits on the skin, in Handbook of diet, nutrition and the skin. 2012, Springer Life Sciences p. 145-164.
7. The useful of topical application of lipophilic anhydrous gel rich in vitamin E. Journal of the American Academy of Dermatology, 2014. 70(5): p. AB197.
8. Rachel, H., Vitamin E and wound healing: an evidence-based review. International Wound Journal, 2016. 13(3): p. 331-335.
9. Galeano, M., et al., Raxofelast, a hydrophilic vitamin E-like antioxidant, stimulates wound healing in genetically diabetic mice. Surgery, 2001. 129(4): p. 467-477.
10. Musalmah, M., et al., Comparative effects of palm vitamin E and alpha-tocopherol on healing and wound tissue antioxidant enzyme levels in diabetic rats. Lipids, 2005. 40(6): p. 575-80.
11. Jervis, K. M. and B. Robaire, The effects of long-term vitamin E treatment on gene expression and oxidative stress damage in the aging Brown Norway rat epididymis. Biol Reprod, 2004. 71(4): p. 1088-95.
12. Molina-Rubio, M. P., N. B. Casas-Alencáster, and L. P. Martinez-Padilla, Effect of formulation and processing conditions on the rheological and textural properties of a semi-liquid syrup model. Food Research International, 2010. 43(3): p. 678-682.
13. Simerdeep Singh Gupta, A. M., Tapan Parikh, Abu T. M. Serajuddin, Investigation of thermal and viscoelastic properties of polymers relevant to hot melt extrusion-I: Polyvinylpyrrolidone and related polymers. Journal of Excipients & Food Chemicals, 2014. 5 (1): p. 32-45.
14. Jones, D. S., et al., Rheological, mechanical and mucoadhesive properties of thermoresponsive, bioadhesive binary mixtures composed of poloxamer 407 and carbopol 974P designed as platforms for implantable drug delivery systems for use in the oral cavity. Int J Pharm, 2009. 372(1-2): p. 49-58.
15. Pereira, R. R., et al., Preparation and characterization of mucoadhesive thermoresponsive systems containing propolis for the treatment of vulvovaginal candidiasis. J Pharm Sci, 2013. 102(4): p. 1222-34.
16. Sachdeva, J. S., Evaluation of Silicate and Polymer Systems for Disproportionate Permeability Reduction in Oil Reservoirs. 2015, University of Stavanger. p. 159.
17. Mezger, T. G., The Rheology Handbook: For Users of Rotational and Oscillatory Rheometers. 2011, Hanover, Germany: European coatings tech files.
18. Wan, T., et al., Strain amplitude response and the microstructure of PA/clay nanocomposites. Polymer, 2005. 46(17): p. 6429-6436.
19. Suwardie, H., et al., Rheological study of the mixture of acetaminophen and polyethylene oxide for hot-melt extrusion application. European Journal of Pharmaceutics and Biopharmaceutics, 2011. 78(3): p. 506-512.
20. Carvalho, F. C., et al., Rheological, mechanical, and bioadhesive behavior of hydrogels to optimize skin delivery systems. Drug Dev Ind Pharm, 2013. 39(11): p. 1750-7.
21. Tamburic, S. and D. Q. M. Craig, A comparison of different in vitro methods for measuring mucoadhesive performance. European Journal of Pharmaceutics and Biopharmaceutics, 1997. 44(2): p. 159-167.
22. Bonacucina, G., S. Martelli, and G. F. Palmieri, Rheological, mucoadhesive and release properties of Carbopol gels in hydrophilic cosolvents. International Journal of Pharmaceutics, 2004. 282(1): p. 115-130.
23. Hurler, J., et al., Improved texture analysis for hydrogel characterization: Gel cohesiveness, adhesiveness, and hardness. Journal of Applied Polymer Science, 2012. 125(1): p. 180-188.
24. Baloglu, E., et al., Rheological and mechanical properties of poloxamer mixtures as a mucoadhesive gel base. Pharm Dev Technol, 2011. 16(6): p. 627-36.
25. Gratieri, T., et al., A poloxamer/chitosan in situ forming gel with prolonged retention time for ocular delivery. Eur J Pharm Biopharm, 2010. 75(2): p. 186-93.
26. Tugcu-Demiroz, F., F. Acarturk, and D. Erdogan, Development of long-acting bioadhesive vaginal gels of oxybutynin: formulation, in vitro and in vivo evaluations. Int J Pharm, 2013. 457(1): p. 25-39.
27. Andrews, G. P. and D. S. Jones, Rheological characterization of bioadhesive binary polymeric systems designed as platforms for drug delivery implants. Biomacromolecules, 2006. 7(3): p. 899-906.
28. Jones, D. S., A. D. Woolfson, and J. Djokic, Texture profile analysis of bioadhesive polymeric semisolids: Mechanical characterization and investigation of interactions between formulation components. Journal of Applied Polymer Science, 1996. 61(12): p. 2229-2234.
29. Tuğcu-Demiröz, F., F. Acarturk, and D. Erdoğan, Development of long-acting bioadhesive vaginal gels of oxybutynin: Formulation, in vitro and in vivo evaluations. International Journal of Pharmaceutics, 2013. 457(1): p. 25-39.
30. De Souza Ferreira, S. B., et al., Rheological, mucoadhesive and textural properties of thermoresponsive polymer blends for biomedical applications. J Mech Behav Biomed Mater, 2015. 55: p. 164-78.
31. Tamburic, S. and D. Q. M. Craig, Rheological Evaluation of Polyacrylic Acid Hydrogels. Pharmacy and Pharmacology Communications, 1995. 1(3): p. 107-109.
32. Cevher, E., et al., Evaluation of mechanical and mucoadhesive properties of clomiphene citrate gel formulations containing carbomers and their thiolated derivatives. Drug Deliv, 2008. 15(1): p. 57-67.
33. Karavana, S. Y., P. Guneri, and G. Ertan, Benzydamine hydrochloride buccal bioadhesive gels designed for oral ulcers: preparation, rheological, textural, mucoadhesive and release properties. Pharm Dev Technol, 2009. 14(6): p. 623-31.

### REFERENCES—EXAMPLE 2

Ahn, T. S., Lee, J.-P., Kim, J., Oh, S. Y., Chun, M.-K., Choi, H.-K., 2013. Effect of pressure sensitive adhesive and vehicles on permeation of terbinafine across porcine hoof membrane. Archives of Pharmacal Research 36, 1403-1409.
Anders, K., Lee, G., 2015. Effect of residual solvent in polymer adhesive matrix on release and skin permeation of scopolamine. International Journal of Pharmaceutics 491, 42-48.
Aulton, M., Cole, G., Hogan, J., 1995. Pharmaceutical Coating Technology. Taylor & Francis.
Banker, G. S., 1966. Film coating theory and practice. Journal of Pharmaceutical Sciences 55, 81-89.
Baskar, D., Sampath Kumar, T. S., 2009. Effect of deacetylation time on the preparation, properties and swelling behavior of chitosan films. Carbohydrate Polymers 78, 767-772.
Blanco-Fernandez, B., Rial-Hermida, M. I., Alvarez-Lorenzo, C., Concheiro, A., 2013. Edible chitosan/acetylated monoglyceride films for prolonged release of vitamin E and antioxidant activity. Journal of Applied Polymer Science 129, 626-635.
Boateng, J. S., Stevens, H. N. E., Eccleston, G. M., Auffret, A. D., Humphrey, M. J., Matthews, K. H., 2009. Development and mechanical characterization of solvent-cast polymeric films as potential drug delivery systems to mucosal surfaces. Drug Development and Industrial Pharmacy 35, 986-996.
Boscoboinik, D., Szewczyk, A., Hensey, C., Azzi, A., 1991. Inhibition of cell proliferation by alpha-tocopherol. Role of protein kinase C. Journal of Biological Chemistry 266, 6188-6194.
Cassano, R., 2012. Vitamin E chemistry, biological activity and benefits on the skin, Handbook of diet, nutrition and the skin. Springer Life Sciences pp. 145-164.
Dirim, S. N., Özden, H. Ö., Bayindirli, A., Esin, A., 2004. Modification of water vapour transfer rate of low density polyethylene films for food packaging. Journal of Food Engineering 63, 9-13.
Felton, L. A., O'Donnell, P. B., McGinity, J. W., 2008. Mechanical properties of polymeric films prepared from aqueous dispersions, Aqueous Polymeric Coatings for Pharmaceutical Dosage Forms, Third Edition. CRC Press, pp. 125-148.
Henkel, 2013. DURO-TAK and GELVA Transdermal Pressure Sensitive Adhesives; Product Selector Guide. http://www.pressure-sensitive-solutions.com/content/dam/uai/AIH/master/images/drug-delivery-polymers/transdermal-adhesives-guide.pdf.
Hermans, K., Van den Plas, D., Kerimova, S., Carleer, R., Adriaensens, P., Weyenberg, W., Ludwig, A., 2014. Development and characterization of mucoadhesive chitosan films for ophthalmic delivery of cyclosporine A. International Journal of Pharmaceutics 472, 10-19.
Hsu, S.-T., Lawrence Yao, Y., 2014. Effect of Film Formation Method and Annealing on Morphology and Crystal Structure of Poly(L-Lactic Acid) Films.
Jog, R., Gokhale, R., Burgess, D. J., 2016. Solid state drug-polymer miscibility studies using the model drug ABT-102. International Journal of Pharmaceutics 509, 285-295.
Jung, E., Lee, E. Y., Choi, H.-K., Ban, S.-J., Choi, S.-H., Kim, J. S., Yoon, I.-S., Kim, D.-D., 2015. Development of drug-in-adhesive patch formulations for transdermal delivery of fluoxetine: In vitro and in vivo evaluations. International Journal of Pharmaceutics 487, 49-55.
Kaji, M., Takeyama, Y., Nioh, A., Tsuyuki, M., Akatsuka, H., Fujiwara, S., Sakai, K., Sakai, H., 2017. Surface Morphology of Cosmetic Film Consisting of PEG-Diisostearate Amphiphilic Random Copolymer, Xanthan Gum, and Solvents. Journal of Oleo Science advpub.
Karki, S., Kim, H., Na, S.-J., Shin, D., Jo, K., Lee, J., 2016. Thin films as an emerging platform for drug delivery. Asian Journal of Pharmaceutical Sciences 11, 559-574.
Lim, H., Hoag, S. W., 2013. Plasticizer Effects on Physical-Mechanical Properties of Solvent Cast Soluplus® Films. AAPS PharmSciTech 14, 903-910.
Michaelis, M., Brummer, R., Leopold, C. S., 2014. Plasticization and antiplasticization of an acrylic pressure sensitive adhesive by ibuprofen and their effect on the adhesion properties. European Journal of Pharmaceutics and Biopharmaceutics 86, 234-243.
Ofori-Kwakye, K., Fell, J. T., 2003. Biphasic drug release from film-coated tablets. International Journal of Pharmaceutics 250, 431-440.
Salawi, A., Nazzal, S., 2018. The rheological and textural characterization of Soluplus®/Vitamin E composites. International Journal of Pharmaceutics 546, 255-262.
Schulz, M., Fussnegger, B., Bodmeier, R., 2010. Drug release and adhesive properties of crospovidone-containing matrix patches based on polyisobutene and acrylic adhesives. European Journal of Pharmaceutical Sciences 41, 675-684.
Shamma, R. N., Basha, M., 2013. Soluplus®: A novel polymeric solubilizer for optimization of Carvedilol solid dispersions: Formulation design and effect of method of preparation. Powder Technology 237, 406-414.
Thakhiew, W., Devahastin, S., Soponronnarit, S., 2013. Physical and mechanical properties of chitosan films as affected by drying methods and addition of antimicrobial agent. Journal of Food Engineering 119, 140-149.
Tuntiyasawasdikul, S., Limpongsa, E., Jaipakdee, N., Sripanidkulchai, B., 2015. A monolithic drug-in-adhesive patch of methoxyflavones from Kaempferia parviflora: In vitro and in vivo evaluation. International Journal of Pharmaceutics 478, 486-495.

1. A composition comprising a polyvinyl caprolactam-polyvinyl acetate-polyethylene glycol graft copolymer (PCL-PVAc-PEG) and a partially hydrophilic oil, wherein the composition transitions from a viscous liquid, to an adhesive patch, and to a film as a weight percent (wt %) ratio of PCL-PVAc-PEG to partially hydrophilic oil changes.

2. The composition of claim 1, wherein transition between the viscous liquid to the adhesive patch occurs when the PCL-PVAc-PEG comprises about 25 to 50 wt % and the partially hydrophilic oil comprises about 50 to 75 wt % of the composition; or wherein the transition between the adhesive and the film occurs when the PCL-PVAc-PEG comprises about 50 to less than 100 wt % and the partially hydrophilic oil comprises greater than 0% to 50 wt % of the composition.

3. (canceled)

4. The composition of claim 1, wherein the film has at least one of: a tensile strength with a range from 0.01 MPa to 6 MPa; an elasticity with a range of 50 MPa to 4500 MPa Young's modulus; or an adhesiveness with an adhesive range from 6 g to 540 g of force.

5. (canceled)

6. (canceled)

7. The composition of claim 1, wherein the PCL-PVAc-PEG has a molecular weight in the range of 90,000-140,000 g/mol.

8. The composition of claim 1, wherein the partially hydrophilic oil has a free hydroxyl group or is at least one of vitamin E, eugenol, clove oil, or black seed oil.

9. (canceled)

10. The composition of claim 1, further comprising at least one of a polar solvent or an active agent.

11. (canceled)

12. The composition of claim 1, further comprising depositing or molding the composition into an adhesive, a film, a composite, an insect or rodent trap, a pressure sensitive adhesive, a transdermal drug delivery patch, a pressure sensitive adhesive, a transdermal drug delivery patch, a film, a pill coating, a wound dressing, a general adhesive, a glue, a food aid, a gummy, an edible film, a face mask, or a soft or a hard gelatin-free capsule.

13. A method of making a composition that transitions between a viscous liquid, an adhesive patch, and a film comprising the steps of:
mixing a acetate-polyethylene glycol graft copolymer (PCL-PVAc-PEG) and a partially hydrophilic oil;
forming a homogeneous mixture; and
depositing an adhesive patch or casting into a film.

14. The method of claim 13, further comprising adding a polar solvent during the mixing step, wherein the polar solvent is one from the group of acetone, ethanol, methanol, and dimethylformamide.

15. The method of claim 13, wherein the step of mixing comprises at least one of sonication, vibration, vortexing, mixing, stirring, shaking, or heating.

16. The method of claim 13, wherein a film is cast by at least one or extrusion, solution casting, or reverse roll coating.

17. The method of claim 13, further comprising removing any bubbles from the mixture.

18. The method of claim 13, wherein transition between the viscous liquid to the adhesive patch occurs when the PCL-PVAc-PEG comprises about 25 to 50 wt % and the partially hydrophilic oil comprises about 50 to 75 wt % of the composition; or wherein the transition between the adhesive and the film occurs when the PCL-PVAc-PEG comprises about 50 to less than 100 wt % and the partially hydrophilic oil comprises greater than 0% to 50 wt % of the composition.

19. (canceled)

20. The method of claim 13, wherein the film has at least one of: a tensile strength with a range from 0.01 MPa to 6 MPa; an elasticity with a range of 50 MPa to 4500 MPa Young's modulus; or an adhesiveness with an adhesive range from 6 g to 540 g of force.

21. (canceled)

22. (canceled)

23. The method of claim 13, wherein the PCL-PVAc-PEG has a molecular weight in the range of 90,000-140,000 g/mol.

24. The method of claim 13, wherein the partially hydrophilic oil has a free hydroxyl group or is at least one of vitamin E, eugenol, clove oil, or black seed oil.

25. (canceled)

26. The method of claim 13, further comprising forming the composition in into an adhesive, a film, a composite, an insect or rodent trap, a pressure sensitive adhesive, a transdermal drug delivery patch, a pressure sensitive adhesive, a transdermal drug delivery patch, a film, a pill coating, a wound dressing, a general adhesive, a glue, a food aid, a gummy, an edible film, a face mask, or a soft or a hard gelatin-free capsule.

27. The method of claim 13, further comprising an active agent.

28. A composition comprising:
a polyvinyl caprolactam-polyvinyl acetate-polyethylene glycol graft copolymer (PCL-PVAc-PEG); and
a partially hydrophilic oil,
wherein the composition transitions from a viscous liquid, to an adhesive, and to a film as a weight percent (wt %) ratio of PCL-PVAc-PEG to partially hydrophilic oil changes, the transition between the viscous liquid to the adhesive occurs when the PCL-PVAc-PEG comprises about 25 to 50 wt % and the partially hydrophilic oil comprises about 50 to 75 wt % of the composition, and the transition between the adhesive and the film occurs when the PCL-PVAc-PEG comprises about 50 to less than 100 wt % and the partially hydrophilic oil comprises greater than 0% to 50 wt % of the composition.

29. The composition of claim 28, wherein the composition is formed into an adhesive, a film, a composite, an insect or rodent trap, a pressure sensitive adhesive, a transdermal drug delivery patch, a pressure sensitive adhesive, a transdermal drug delivery patch, a film, a pill coating, a wound dressing, a general adhesive, a glue, a food aid, a gummy, an edible film, a face mask, or a soft or a hard gelatin-free capsule.

30. A pressure sensitive adhesive, a transdermal drug delivery patch, a film, or a pill coating composition that transitions between a viscous liquid, an adhesive, and a film made by a method comprising:
mixing a acetate-polyethylene glycol graft copolymer (PCL-PVAc-PEG) and a partially hydrophilic oil;
forming a homogeneous mixture; and
depositing an adhesive or casting solution into a film.
